(19) United States
(12) Patent Application Publication
(10) Pub. No.: US 2004/0185097 A1
(43) Pub. Date: Sep. 23, 2004

(54) Controlled release modifying complex and pharmaceutical compositions thereof

(75) Inventors: Muthaiyyan Esakki Kannan, Mumbai (IN); Anandi Krishnan, Mumbai (IN); Beena Amol Sapre, Mumbai (IN); Chitra Siddharth Shah, Mumbai (IN); Atul Vishvanath Patil, Mumbai (IN)

Correspondence Address: LERNER, DAVID, LITTENBERG,, KRUMHOLZ & MENTLIK, 600 SOUTH AVENUE WEST, WESTFIELD, NJ, 07090, US

(73) Assignee: Glenmark Pharmaceuticals Ltd., Princeton, NJ

(21) Appl. No.: 10/762,180

(22) Filed: Jan. 21, 2004

Related U.S. Application Data
(60) Provisional application No. 60/517,589, filed on Nov. 5, 2003.

(30) Foreign Application Priority Data
Jan. 31, 2003 (IN) 130/MUM/2003

(51) Int. Cl. A61K 9/22

(52) U.S. Cl. 424/468

(57) ABSTRACT

Disclosed is a controlled release modifying complex for solid oral controlled release pharmaceutical compositions suitable for once-a-day administration. The composition comprises an active pharmaceutical ingredient, a release modifying complex and other required pharmaceutically acceptable excipients. The release modifying complex comprises a primary release modifying agent, a secondary release modifying agent and an auxiliary release modifying agent or varying combinations thereof, wherein said primary, secondary and auxiliary release modifying agents are present in amounts that synergistically effect and extend the release of active pharmaceutical ingredient.

### CROSS REFERENCE TO RELATED APPLICATIONS

This patent application claims the benefit of the filing date of the U.S. Provisional Application No. 60/517,589, filed Nov. 5, 2003, and the priority from the filing date of the Indian Provisional Application No. 132/Mum/2003, filed Jan. 31, 2003, the disclosures of both of which are hereby incorporated by reference in their entirety.

### BACKGROUND OF THE INVENTION

Traditionally, solid oral pharmaceutical dosage forms (drug delivery systems) are comprised of immediate release (IR) dosages in the form of tablets or capsules. These IR dosage forms release the active drug substance into the body of a subject at a rate that is initially very high followed by a rapid decline. One potential result of an IR dosage form is that the subject may have varying degrees of blood level fluctuation, which may result in transient therapeutic overdose, followed by a period of therapeutic under dosing. These blood level fluctuations are known as “peaks and valleys” or similarly as “peaks and troughs.”

One of the most frequently utilized methods to extend the duration of drug action in the body and/or control blood level fluctuations is modification of the pharmaceutical dosage form. This is usually achieved with single or multi-component matrix systems such as granules, pellets, tablets or a combination of the above where the drug delivery is mainly controlled by a diffusion, osmotic or erosion mechanism.

Controlled-release (CR) formulations have the advantage that the active drug is gradually released over a relatively long period so that the drug is maintained in the blood stream for a longer time and at a more uniform concentration than would otherwise be the case. This allows administration only once or twice daily for drugs that would otherwise have to be taken more frequently to maintain required blood levels. Many different types of controlled-release oral dosage forms have been developed, but each has disadvantages, which affect its suitability to a particular drug and therapeutic objective.

The most common Immediate Release (IR) oral dosage forms are administered more than once a day. These IR dosage forms may lead to a peak and trough blood level fluctuations. The more than once daily dosing can also result in a poor compliance due to its multiple dosing regimen. The most effective approach to overcome the above mentioned non-compliance causes have been to develop a controlled release oral solid pharmaceutical composition of clarithromycin.

U.S. Pat. No. 4,389,393 discloses a sustained release therapeutic compositions based on high molecular weight hydroxypropyl methylcellulose. A carrier base material combined with a therapeutically active medicament and shaped and compressed to a solid unit dosage form having a regular and prolonged release pattern upon administration.

U.S. Pat. No. 4,540,566 discloses a prolonged release drug dosage forms based on modified low viscosity grade hydroxypropyl methylcellulose. A carrier base material combined with a therapeutically active medicament and shaped and compressed to a solid unit dosage form having a controlled and prolonged release pattern upon administration.

U.S. Pat. No. 5,393,765 describes an erodible pharmaceutical composition providing a unique zero order controlled release profile. The erodible composition contains a therapeutically active substance having solubility not greater than 80 mg/mL, a hydroxypropyl methylcellulose derivative and erosion modifiers depending on drug solubility and drug loading, such as lactose and polyoxyalkylene derivatives of propylene glycol, as well as other inert materials such as binders and lubricants.

U.S. Pat. No. 6,010,718 discloses an extended release formulation of erythromycin derivatives. The formulation comprises an erythromycin derivative and a pharmaceutically acceptable polymer so that, when ingested orally, the composition induces significantly lower Cmax in the plasma than an immediate release composition of the erythromycin derivative while maintaining bioavailability and minimum concentration substantially equivalent to that of the immediate release composition of the erythromycin derivative upon multiple dosing. The compositions of the invention have an improved taste profile and reduced gastrointestinal side effects as compared to those for the immediate release composition.

U.S. Pat. No. 5,705,190 is directed to a controlled release, oral, solid, pharmaceutical composition for a reduced daily dosage regimen, where the therapeutic ingredient is a poorly soluble basic drug. The formulation comprises the use of a water-soluble alginate salt, a complex salt of alginic acid and an organic carboxylic acid in admixture with the therapeutic drug.

U.S. Pat. No. 4,808,411 describes compositions, which comprise a complex of carbomer (acrylic acid polymers) and erythromycin or a derivative thereof such as 6-O-methylerythromycin. The compositions provide nontoxic, palatable dry and liquid dosage forms for oral administration.

The drug delivery systems disclosed in these patents provide for solid, oral controlled release dosage forms of Active Pharmaceutical Ingredients (API). However, these systems contain additional disadvantages such as relatively fast drug release profiles, dose dumping, lack of stability, variation in drug release profile between the units and difficult or expensive manufacturing methods.

Therefore, there still exists a need for developing an efficient controlled release pharmaceutical composition of an API that is capable of controlled drug delivery in order to prevent and improve upon these problems.

### SUMMARY OF THE INVENTION

The present invention relates to a controlled release modifying complex for solid oral controlled release pharmaceutical compositions suitable for once-a-day administration. More particularly, the pharmaceutical composition comprises an API, a release modifying complex and other required pharmaceutically acceptable excipients. The release modifying complex of the invention comprises a primary, secondary and auxiliary release modifying agent or combination thereof, wherein said primary, secondary and auxiliary release modifying agents are present in amounts that synergistically effect and extend the release of the erythromycin derivative.

It has been discovered that the controlled-release (CR) formulations of the present invention which comprises an active pharmaceutical ingredient (“API”) and a controlled release modifying complex, wherein said release modifying complex synergistically effects and extends the release of the API and thereby prevents dose dumping and unwanted plasma level fluctuations.

In one aspect, the present invention relates to a CR pharmaceutical composition of an API or their pharmaceutically acceptable salts, ester or hydrates, compressible to a size suitable for oral administration to humans, comprising an API and a controlled release modifying complex, so that when ingested orally, the API is released into the gastrointestinal tract predominantly in solution phase rather than a solid phase thus avoiding any chances of dose dumping and thereby reduce the drug release variation between dosage units to a minimum.

In another aspect, the present invention relates to a CR pharmaceutical composition comprising an API or their pharmaceutically acceptable salts, ester or hydrates and a controlled release modifying hydrophilic complex, so that when ingested orally, the complex slowly releases the API over an extended period of time so as to provide a therapeutic effective level and plasma concentration of the API that is suitable for once-a-day dosing.

In another aspect, the present invention relates to a CR pharmaceutical composition comprising a macrolide or azalide antibiotic or their pharmaceutically acceptable salts, ester or hydrates and a controlled release modifying complex, so that when ingested orally, the complex slowly releases said macrolide or azalide over an extended period of time so as to provide a therapeutic effective level and plasma concentration of said macrolide or azalide that is suitable for once-a-day dosing.

In another aspect, the present invention relates to a CR pharmaceutical composition comprising a quinolone antibiotic or its pharmaceutically acceptable salts, ester or hydrates and a controlled release modifying complex, so that when ingested orally, the complex slowly releases said quinolone over an extended period of time so as to provide a therapeutic effective level and plasma concentration of said quinolone that is suitable for once-a-day dosing.

In another aspect, the present invention relates to a CR pharmaceutical composition comprising a cephalosporin antibiotic or its pharmaceutically acceptable salts, ester or hydrates and a controlled release modifying complex, so that when ingested orally, the complex slowly releases said cephalosporin antibiotic over an extended period of time so as to provide a therapeutic effective level and plasma concentration of said cephalosporin antibiotic that is suitable for once-a-day dosing.

In another aspect, the present invention relates to a CR pharmaceutical composition comprising a penicillin antibiotic or its pharmaceutically acceptable salts, ester or hydrates and a controlled release modifying complex, so that when ingested orally, the complex slowly releases said penicillin antibiotic over an extended period of time so as to provide a therapeutic effective level and plasma concentration of said penicillin antibiotic that is suitable for once-a-day dosing.

In another aspect, the present invention relates to a CR pharmaceutical composition comprising a high soluble high dose API or their pharmaceutically acceptable salts, ester or hydrates and a controlled release modifying complex, so that when ingested orally, the complex slowly releases said API over an extended period of time so as to provide a therapeutic effective level and plasma concentration of said API that is suitable for once-a-day dosing.

In another aspect, the present invention relates to a CR pharmaceutical composition comprising a high soluble low dose API or their pharmaceutically acceptable salts, ester or hydrates and a controlled release modifying complex, so that when ingested orally, the complex slowly releases said API over an extended period of time so as to provide a therapeutic effective level and plasma concentration of said API that is suitable for once-a-day dosing.

In another aspect, the present invention relates to a CR pharmaceutical composition comprising a low soluble high dose API or their pharmaceutically acceptable salts, ester or hydrates and a controlled release modifying complex, so that when ingested orally, the complex slowly releases said API over an extended period of time so as to provide a therapeutic effective level and plasma concentration of said API that is suitable for once-a-day dosing.

In another aspect, the present invention relates to a CR pharmaceutical composition comprising a low soluble low dose API or their pharmaceutically acceptable salts, ester or hydrates and a controlled release modifying complex, so that when ingested orally, the complex slowly releases said API over an extended period of time so as to provide a therapeutic effective level and plasma concentration of said API that is suitable for once-a-day dosing.

In yet another aspect, the present invention relates to a CR pharmaceutical composition comprising an API and a controlled release modifying complex, so that when ingested orally, the composition provides a convenient, generally self-administered dosage form that yields a constant infusion of the drug. Advantages of the controlled release drug delivery system of the present invention include, but are not limited to: (1) Reduction in drug blood level fluctuations. For example, by controlling the rate of drug release, “peaks and valleys” of drug-blood or serum levels are eliminated. (2) Reduction in dosing frequency. For example, rate-controlled products deliver more than a single dose of medication and thus are taken less often than conventional forms. (3) Enhanced patient convenience and compliance. For example, with less frequency of dose administration, the patient is less apt to neglect taking a dose. There is also greater patient convenience with daytime and nighttime medication, and control of chronic illness. (4) Reduction in adverse side effects. Because there are seldom drug blood level peaks above the drug's therapeutic range, and into the toxic range, adverse side effects are less frequently encountered.

In another aspect, the present invention relates to a controlled release pharmaceutical composition of an API comprising an API and a synergistic release modifying complex wherein said complex comprises, (a) a primary release modifying agent, (b) a secondary release modifying agent, and (c) an auxiliary release modifying agent, so that when ingested orally, said complex synergistically effects and extends release of the API.

In another aspect, the present invention relates to a controlled release pharmaceutical composition of an API comprising an API and a synergistic release modifying complex wherein said complex comprises, (a) a primary release modifying agent, or (b) a secondary release modifying agent, and (c) an auxiliary release modifying agent, so that when ingested orally, said complex synergistically effects and extends release of the API.

In yet still another aspect, the present invention relates to a controlled release pharmaceutical composition of an API comprising an API and a synergistic release modifying complex, wherein said complex comprises, (a) a primary release modifying agent selected from low molecular weight hydrophilic polymers, (b) a secondary release modifying agent selected from high molecular weight hydrophilic polymers, and (c) an auxiliary release modifying agent selected from starch derivatives.

In yet still another aspect, the present invention relates to a controlled release pharmaceutical composition of an API comprising an API and a synergistic release modifying complex, wherein said complex comprises, (a) a primary release modifying agent selected from low molecular weight hydrophilic polymers, or (b) a secondary release modifying agent selected from high molecular weight hydrophilic polymers, and (c) an auxiliary release modifying agent selected from starch derivatives.

The pharmaceutical composition of the invention also relates to a wide variety of API's suitable for use in controlled release formulations.

The present invention also relates to a process, for the preparation of a controlled release composition of an API suitable for once-a-day administration, comprising a wet granulation, dry granulation, slugging, roll compaction, direct compression or any other technique known in the pharmaceutical art.

### BRIEF DESCRIPTION OF THE DRAWINGS

FIG. 1: illustrates a plasma concentration profile of clarithromycin 500 mg extended release tablets of Example 13.

### DEFINITIONS

The term “controlled release” as used herein means a drug dosage system in which the rate of the API release is more precisely controlled compared to that of immediate or sustained release products, wherein the API is delivered from the dosage system at a predictable and predetermined rate within the body of a patient such that a therapeutically effective blood level, devoid of peak and trough fluctuations, is maintained over an extended period of time.

The term “drug delivery systems” as used herein means the technology utilized to present the drug to the desired body site for drug release and absorption.

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by local or by systemic administration of the active ingredient to the host.

By “pharmaceutically acceptable” is meant those salts and esters which are, within the scope of sound medical judgment, suitable for use in contact with the tissues of humans and lower animals without undue toxicity, irritation, allergic response and the like, commensurate with a reasonable benefit/risk ratio, and effective for their intended use. Representative acid additions salts include the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarare, succinate, tartrate, ascorbate, glucoheptonate, lactobionate, lauryl sulphate salts and the like. Representative alkali or alkaline earth metal salts include the sodium, calcium, potassium and magnesium salts, and the like.

The term “subject” or “a patient” or “a host” as used herein refers to mammalian animals, preferably human.

The term “high soluble API” as used herein will mean that less than 30 parts of water is required to completely dissolve 1 part of the API.

The term “low soluble API” as used herein will mean that greater than 30 parts of water is required to completely dissolve 1 part of the API.

The term “high dose API” as used herein will mean that the individual unit dose of the API is 50 mg or greater.

The term “low dose API” as used herein will mean that the individual unit dose is less than 50 mg.

The term “high soluble high dose API” as used herein will mean that less than 30 parts of water is required to completely dissolve 1 part of the API and that the individual unit dose of the API is 50 mg or greater.

The term “high soluble low dose API” as used herein will mean that less than 30 parts of water is required to completely dissolve 1 part of the API and that the individual unit dose of the API is less than 50 mg.

The term “low soluble high dose API” as used herein will mean that greater than 30 parts of water is required to completely dissolve 1 part of the API and that the individual unit dose of the API is 50 mg or greater.

The term “low soluble low dose API” as used herein will mean that greater than 30 parts of water is required to completely dissolve 1 part of the API and that the individual unit dose of the API is less than 50 mg.

### DETAILED DESCRIPTION OF THE INVENTION

The present invention relates to a solid oral controlled release modifying complex and pharmaceutical compositions thereof for once-a-day administration to a subject in need thereof. More particularly, the pharmaceutical composition comprises an API, a controlled release modifying complex and other required pharmaceutically acceptable excipients. The controlled release modifying complex of the invention comprises a primary, secondary and/or auxiliary release modifying agent or varying combination thereof, wherein said primary, secondary and auxiliary release modifying agents are present in amounts that synergistically effect and extend the release of the API.

The pharmaceutical composition of the present invention shows a predictable and predetermined controlled drug release, so that the API is available over a period up to 24 hours. The release period is dependent on the precise tablet size, the identity of the active ingredient, aqueous solubility of the active ingredient, hardness and particular composition of the release modifying complex. The composition prepared in accordance with the present invention is hard, has low friability and provides controlled release over an extended period. Moreover, the drug release profile of each dosage unit is uniform and without any significant variation.

According to the present invention, a preferred API is a macrolide or azalide antibiotic. Preferably, the API is an erythromycin derivative selected from the group consisting of josamycin, midecamycin, kitamycin, roxithromycin, rokitamycin, oleandomycin, miocamycin, flurithromycin, rosaramicin, azithromycin, and clarithromycin, and their pharmaceutically acceptable hydrates, salts and esters.

In another embodiment of the present invention, a preferred API is a penicillin class antibiotic or derivative thereof. Preferably, the API is a penicillin selected from the group consisting of Amoxicillin, Ampicillin, Ampicillin Sodium, Apalcillin, Aspoxicillin, Azlocillin, Aztreonam, Bacampicillin, Cabenicillin, Carfecillin, Carindacillin, Ciclacillin, Cloxacillin, Dicloxacillin, and their pharmaceutically acceptable hydrates, salts and esters.

In yet another embodiment of the present invention, a preferred API is a cephalosporin class antibiotic or derivative thereof. Preferably, the API is a cephalosporin selected from the group consisting of Cefacetrile, Cefadroxil, Cefaloridine, Cefalothin Sodium, Cefapirin, Cefazaflur, Cefazedone, Cefazolin, Cefradine, Ceftezole, Cefsulodin Sodium, Cefamandole, Cefonicid, Cefoperazone, Cefuroxime, Cefuzonam, Cefbuperazone, Cefoxitin, Cefminox, Cefinetazole, Cefotetan, Loracarbef, Cefinenoxime, Cefodizime Sodium, Cefotaxime, Cefpimizole, Cefpiramide, Ceftazidime, Ceftiolene, Ceftizoxime, Ceftriaxone, Cefepime, Cefetecol, Cefpirome, Cefquinome, Cefalosporin C, Cefozopran Hydrochloride, Cefaclor, Cefadroxil, Cefalexin, Cefaloglycine, Cefatrizine, Cefdinir, Cefetamet Pivoxil, Cefixime, Ceforanide, Cefotiam, Cefpodoxime, Cefpodoxime Proxetil, Cefprozil, Cefradine, Cefroxadine, Cefteram Pivoxil, Ceftibuten, Cefuroxime Axetil, and their pharmaceutically acceptable hydrates, salts and esters.

In still yet another embodiment of the present invention, a preferred API is a quinolone class antibiotic or derivative thereof. Preferably, the API is a quinolone selected from the group consisting of Nalidixic Acid, Cinoxacin, Oxolinic Acid, Flumequine, Pipemidic Acid, Rosoxacin, Norfloxacin, Lomefloxacin, Ofloxacin, Enrofloxacin, Ciprofloxacin, Enoxacin, Amifloxacin, Fleroxacin, Gatifloxacin, Gemifloxacin, Pefloxacin, Rufloxacin, Sparfloxacin, Temafloxacin, Tosufloxacin, Grepafloxacin, Levofloxacin, Moxifloxacin, Trovafloxacin, and their pharmaceutically acceptable hydrates, salts and esters.

In still yet another embodiment of the present invention, a preferred API is a high soluble high dose API or derivative thereof. Preferably, the API is a high soluble high dose API selected from the group consisting of Acebutolol hydrochloride, Amantadine hydrochloride, Aminocaproic acid, Aminophylline, Amodiaquine hydrochloride, Ascorbic acid, Carbenoxolone sodium, Cefuroxime sodium, Chloroquine phosphate, Chloroquine sulphate, Chlorpromazine hydrochloride, Ciprofloxacin hydrochloride, Cloxacillin sodium, Cycloserine, Diltiazem hydrochloride, Diethyl carbamazine citrate, Doxycycline hydrochloride, Ethosuximide, Ferrous gluconate, Isoniazid, Levamisole hydrochloride, Lincomycin hydrochloride, Mebeverine hydrochloride, Mepyramine maleate, Metformin hydrochloride, Metoprolol tartrate, Nicotinamide, Nicotinic acid, Oxprenolol hydrochloride, Oxytetracycline hydrochloride, Penicillamine, Pentobarbitone sodium, Phenoxy Methyl Penicillin K, Phenyloin sodium, piperazine adipate, Procainamide hydrochloride, Pseudoephedrine hydrochloride, Quinalbarbitone sodium, Quinine bisulphate, Ranitidine hydrochloride, Sodium amino salicylate, Sodium fusidate, sodium valproate, Streptomycin sulphate, Tetracycline hydrochloride, Troxidone, Verapamil hydrochloride and the like and their pharmaceutically acceptable salts, ester and hydrates.

In still yet another embodiment of the present invention, a preferred API is a high soluble low dose API or derivative thereof. Preferably, the API is a high soluble low dose API selected from the group consisting of Amitriptylline hydrochloride, captopril, Clonidine hydrochloride, Colchicin, Cyclophosphamide, Diphenhydramine hydrochloride, Dothiepen hydrochloride, Doxepin hydrochloride Ephedrine hydrochloride, Ergometrine maleate, Ergometrine tartrate, Fenfluramine hydrochloride, Folic acid, Glyceryl trinitrate, Hyoscine hydrobromide, Hyoscine buytlbromide, Imipramine hydrochloride, Isoprenaline sulphate, Isosorbide dinitrate, Isosorbide-5-mononitrate, Methadone hydrochloride, Methdilazine hydrochloride, Metoclopramide hydrochloride, Neostigmine bromide, Oxybutynin hydrochloride, Pethidine hydrochloride, Phenformin hydrochloride, Pheniramine maleate, Phenobarbitone sodium, Primaquine phosphate, Promethazine hydrochloride, Propantheline bromide, Propranolol hydrochloride, Pyridoxine hydrochloride, Salbutamol sulphate, Terbutaline sulphate, Thiamine hydrochloride, Timolol maleate, Trifluoperazine hydrochloride, Triflupromazine hydrochloride, Triprolidine hydrochloride, Warfarin sodium and the like and their pharmaceutically acceptable salts, ester and hydrates.

In still yet another embodiment of the present invention, a preferred API is a low soluble high dose API or derivative thereof. Preferably, the API is a low soluble high dose API selected from the group consisting of Acetazolamide, Allopurinol, Atenolol, Carbamazepine, Cefadroxil, Cephalexin, Chloramphenicol, Cefuroxime Axetil, Chlorthalidone, Cimetidine, Clarithromycin, Clofazemine, Dapsone, Diclofenac sodium, Diiodohydroxy quinolone, Diloxanide furoate, Disulfiram, Erythromycin, Erythromycin estolate, Erythromycin stearate, Ethacrynic cid, Ethionamide, Ethopropazine hydrochloride, Ferrous fumarate, Fluconazole, Flurbiprofen, Furazolidone, Griseofulvin, Hydrochlorthiazide, Ibuprofen, Ketoconazole, Ketoprofen, Labetalol hydrochloride, Levodopa, Linezolid, Lithium carbonate, Magaldrate, Mebendazole, Mefenamic acid, Megestrol acetate, Mercaptopurine, Nalidixic acid, Niclosamide, Nitrofurantoin, Norfloxacin, Oxyphenbutazone, Paracetamol, Phenindione, Phenobarbitone, Phenylbutazone, Phenylsulphathiazole, piperazine phosphate, Proguanil hydrochloride, Promethazine theoclate, Propylthiouracil, Quinidine sulphate, Quinine sulphate, Quinidochlor, Rifampicin, Spironolactone, Succinylsulphathiazole, Sulphadiazine, Sulphadimethoxine, Sulphadimidine, Sulphafurazole, Sulphaphenazole, Thiabendazole, Tinidazole, Tolbutamide, Triamterene, Sulphamethoxazole and the like and their pharmaceutically acceptable salts, ester and hydrates.

In still yet another embodiment of the present invention, a preferred API is a low soluble low dose API or derivative thereof. Preferably, the API is a low soluble low dose API selected from the group consisting of Alprazolam, Amiloride hydrochloride, Astemizole, Benzhexol hydrochloride, Betamethasone, Bromhexine hydrochloride, Bromocryptine mesylate, Buprenorphine hydrochloride, carbimazole, Chlordiazepoxide, Cortisone acetate, cyproheptadine hydrochloride, diazepam, Dexamethasone hydrochloride, Dextromethorphan hydrochloride, dicyclomine hydrochloride, Dienoestrol, Digitoxin, Digoxin, Dydrogesterone, enalapril maleate, Ethinyloestradiol, Ethyloestrenol, Fludrocortisone acetate, Frusemide, glibenclamide, haloperidol, Indomethacin, Isoxsuprine hydrochloride, Lanatoside C, Lercanidipine hydrochloride, Levonorgesrtel, Lomustine, isoxuprine hydrochloride, methotrexate, Mecizine hydrochloride, Melphalan, Methotrexate, Methylergometrine maleate, Methylprednisolone, Mianserin hydrochloride, Nicoumalone, Nifedipine, Nitrazepam, Norethisterone, nortriptyline hydrochloride, Omeprazole, Ormeloxifene hydrochloride, Pentazocine hydrochloride, Phenindamine tartrate, piroxicam, prazosin hydrochloride, Prednisolone, Prednisone, Prochlorperazine maleate, Riboflavinee, Stilbostrol, Tamoxifen citrate, Thyroxine sodium, triamcinilone, Trimethoprim and the like and their pharmaceutically acceptable salts, ester and hydrates.

In still yet another embodiment of the present invention, the pharmaceutical composition of the invention also relates to a wide variety of API's suitable for use in controlled release formulations. Representative API's may include antibacterial, antacids, analgesic and anti-inflammatory agents, anti-arrhythmic agents, antiprotozoal agents, anti-coagulants, antidepressants, anti-diabetic agents, anti-epileptic agents, antifungal agents, antihistamines, anti-hypertensive agents, anti-muscarnic agents, antineoplastic agents, antimetabolites, anti-migraine agents, anti-Parkinsonian agents, antipsychotic, hypnotic and sedating agents, anti-stroke agents, antitussive, antivirals, cardiac inotropic agents, corticosteroids, disinfectants, diuretics, enzymes, essential oils, gastro-intestinal agents, haemostatics, lipid regulating agents, local anesthetics, opioid analgesics, parasympathomimetics and anti-dementia drugs, peptides and proteins, sex hormones, stimulating agents, vasodilators or mixtures thereof.

The amount of active pharmaceutical ingredient in the composition generally varies from about 0.1% to about 90% by weight of the composition. Preferably, the amount of active pharmaceutical ingredient varies from about 0.1% to about 80% by weight of the composition.

The immediate object of the present invention is to develop an efficient controlled release pharmaceutical composition that is capable of controlled drug delivery of active pharmaceutical ingredient, in order to provide sustained/extended therapeutic effects up to 24 hours without dose dumping. The present invention is directed to an extended release pharmaceutical composition of active pharmaceutical ingredient, said composition comprising a pharmaceutically effective amount of the API, a release modifying complex and the other required pharmaceutically acceptable additives. The said release modifying complex essentially comprises, a primary release modifying agent selected from a low molecular weight polyethylene oxide, a secondary release modifying agent selected from a high molecular weight polyethylene oxide, and an auxiliary release modifying agent selected from a starch derivative. The said release modifying complex when present in a synergistic effective amount is sufficient to extend the release of the active pharmaceutical ingredient.

The release modifying complex of the invention comprises a low molecular weight polyethylene oxide, a high molecular weight polyethylene oxide and a starch derivative. The amount of release modifying complex in the composition of the present invention generally varies from about 1% to about 90% by weight of the composition. Preferably, the amount of said complex varies from about 5% to about 85% by weight of the composition. Most preferably the amount of said complex varies from about 10% to about 80% by weight of the composition.

Polyethylene oxide is a non ionic homopolymer of oxyethylene groups (molecular weight of about 2000 to over 100,000) and they are water soluble. They are thermoplastic agents that are readily calendared, extruded, injection molded or cast. Higher molecular weight polyethylene oxides provide extended release via the hydrophilic matrix approach. Polyethylene oxides on exposure to water or gastric juices hydrate and swell rapidly to form hydrogels with properties ideally suited for controlled release formulations. The Polyethylene oxides are non-ionic, so no interaction between the drug and the polymer is to be expected.

The primary release modifying agent of the present invention is selected from the low molecular weight polyethylene oxides, having a molecular weight of at least 100,000 and the molecular weight range from 100,000 to 900,000. Low molecular weight Polyethylene oxides are commercially available in various grades, under several trade names including Polyox WSR N-10, WSR N-80, WSR N-750, WSR-705, and WSR1105 from The Dow Chemical Co. USA. The different grades under the given trade name represent the differences in oxyethylene contents as well as molecular weight. The pharmaceutical composition of the present invention may contain one low molecular weight polyethylene oxide grade alone or a combination of different grades of low molecular weight polyethylene oxides. All the various grades of polyethylene oxides mentioned above are contemplated to be used in this present invention.

The amount of the primary release modifying agent in the release modifying complex generally varies from about 1% to about 90% by weight of the complex. Preferably, the amount of primary release modifying agent varies from about 5% to about 80% by weight of the complex. Most preferably, the amount of primary release modifying agent varies from about 5% to about 70% by weight of the complex.

The secondary release modifying agent of the present invention is selected from the high molecular weight polyethylene oxides, having a molecular weight of at least 1,000,000 and the molecular weight range from 1,000,000 to 9,000,000. High molecular weight Polyethylene oxides are commercially available in various grades, under several trade names including Polyox WSR N-12K, WSR N-60K, WSR-301, WSR coagulant and WSR-303 from The Dow Chemical Co. USA. The different grades under the given trade name represent the differences in oxyethylene contents as well as molecular weight. The pharmaceutical composition of the present invention may contain one high molecular weight polyethylene oxide grade alone or a combination of different grades of high molecular weight polyethylene oxides. All the various grades of polyethylene oxides mentioned above are contemplated to be used in this present invention.

The amount of the secondary release modifying agent in the release modifying complex generally varies from about 1% to about 95% by weight of the complex. Preferably, the amount of secondary release modifying agent varies from about 5% to about 90% by weight of the complex.

The other essential component of the release modifying complex is the auxiliary release modifying agent, selected from the starch derivatives. Examples of starch derivatives include pregelatinized starch, partially pregelatinized starch and retrograded starch. Pregelatinized starch is a starch that has been chemically and/or mechanically processed to rupture all or a part of the starch granules and so as to render the starch flowable and directly compressible. Partially pregelatinized starch is a physically modified starch having the benefit of a soluble functionality and an insoluble functionality. Partial pregelatinization breaks the bond between the amylase and amylopectin, which are the two polymers, tightly bound in a specific spherocrystalline structure in starch. The partial pregelatinization process results in partial solubility, increased particle size, improved flow properties and compactability.

Retrograded starch is a new pregelatinized starch, which is prepared by enzymatic degradation, precipitation (retrogradation) and washing with ethanol. The retrograded pregelatinized starch is a linear oligosaccharide and is characterized by a high specific surface area. The pharmaceutical composition of the present invention may contain either one of the above starch derivatives alone or a combination of the above starch derivatives as the auxiliary release modifying agents. All the above starch derivatives are contemplated to be used in the present invention. For example, the pharmaceutical composition contemplates the use of retrograded pregelatinized starch. The retrograded pregelatinized starch is commercially available as Prejel PA 5 PH from Avebe Inc. (The Netherlands).

The amount of the auxiliary release modifying agent in the release modifying complex generally varies from about 1% to about 95% by weight of the complex. Preferably, the amount of auxiliary release modifying agent varies from about % to about 95% by weight of the complex. Most preferably, the amount of auxiliary release modifying agent varies from about 10% to about 95% by weight of the complex.

The presence of synergistic effective amounts of the low molecular weight polyethylene oxide and/or the high molecular weight polyethylene oxide in combination with the retrograded starch provides a better controlled drug release profile without dose dumping. When used in the amounts provided, the drug release profile is substantially better than the drug release profile using either low molecular weight polyethylene oxide or high molecular weight polyethylene oxide or retrograded starch alone.

The low molecular weight polyethylene oxide i.e. the primary release modifying agent, the high molecular weight polyethylene oxide i.e., the secondary release modifying agent and the retrograded starch i.e., the auxiliary release modifying agent are present in the pharmaceutical composition of the invention in synergistic effective amounts. When the high molecular weight polyethylene oxide, low molecular weight polyethylene oxide and the retrograded starch are present together as the release modifying complex in the pharmaceutical composition of the invention, the controlled release ability of the composition is better than just an additive effect. More specifically, the pharmaceutical composition of the present invention exhibits a better drug release profile when the release modifying complex comprises the above mentioned components, in the prescribed amounts, than any one by themselves. In another embodiment, the present invention may exhibit a better drug release profile, which is devoid of any dose dumping and also ensures the release of the complete amount of the drug over a period of about 12 to 24 hours depending on the requirement for a particular API. As used herein, the term synergistic effective combination of the above mentioned components when used in combination to effect a better drug release profile or other improved result in the pharmaceutical composition of the present invention relative to the formulation containing one or the other of the three components used individually or formulations containing any other rate controlling, release modifying polymers.

It is very well within the scope of the present invention to achieve a controlled release composition comprising a pharmaceutically active ingredient, release modifying complex and other required pharmaceutically acceptable additives, where the release modifying complex comprises a primary release modifying agent and an auxiliary release modifying agent only. It is also very well within the scope of the present invention to achieve a controlled release composition comprising of the pharmaceutically active ingredient, release modifying complex and other required pharmaceutically acceptable additives, where the release modifying complex comprises a secondary release modifying agent and an auxiliary release modifying agent only.

The pharmaceutical composition of the present invention also contains other required pharmaceutically acceptable excipients. The amount of additional pharmaceutically acceptable excipients generally varies from about 10% to about 90% by weight of the composition.

The pharmaceutically acceptable excipients used in the present invention are selected from the fillers, glidants and lubricants that are typically used in the pharmaceutical arts for oral solid dosage forms. The filler used herein is inert filler, may be water soluble or water insoluble and selected from those typically used in the pharmaceutical art for oral solid dosage forms. Examples include calcium carbonate, dicalcium phosphate, tricalcium phosphate, microcrystalline cellulose, monosaccharide, disaccharides, polyhydric alcohols, sucrose, dextrose, lactose, fructose, mannitol, sorbitol, alone or mixtures thereof and the like and mixtures thereof.

The amount of fillers generally varies from about 1% to about 90% by weight of the composition. All the various fillers mentioned above are contemplated to be used in the present invention.

The glidants of the present invention are selected from those glidants typically used in the pharmaceutical art for oral solid dosage forms. For example, colloidal silicon dioxide, talc alone or mixtures and equivalents thereof. The amount of glidants generally varies from about 0.1% to about 5.0% by weight of the composition.

The lubricants of the present invention are selected from those lubricants typically used in the pharmaceutical art for oral solid dosage forms. For example, stearate salts such as calcium stearate, magnesium stearate, zinc stearate, stearic acid, talc, hydrogenated vegetable oil, vegetable oil derivatives, silica, silicones, high molecular weight poly alkylelene glycol and saturated fatty acids alone or mixtures and equivalents thereof. The amount of lubricants generally varies from about 0.1% to about 5.0% by weight of the composition.

The pharmaceutical composition of the present invention can be optionally coated with a coating, using the polymers or other coating agents which may or may not be intended or designed for the modification of drug release. Examples include cellulose ethers such as ethyl cellulose, hydroxypropyl methyl cellulose, hydroxypropyl cellulose, others such as polyvinyl alcohol, polyvinyl pyrrolidone, methacrylic acid derivatives, resins, clays, long chain hydrocarbons, long chain carboxylic acids, long chain carboxylic acid esters, long chain alcohols and the like or mixtures thereof. The coating composition comprises of the coating polymer and the other required additives like plasticizer, opacifier, colorant, preservatives and the like. The weight gain of the coating generally varies from about 0.1% to about 25.0% by weight of the composition.

The pharmaceutical composition of the present invention may contain other optional ingredients that are also typically used in pharmaceuticals such as coloring agents, preservatives, flavorings, and the like. The amount of optional ingredients generally varies from about 0.1% to about 5.0% by weight of the composition.

Another embodiment of the present invention provides methods of making a controlled release formulation of an active pharmaceutical ingredient by wet granulation, dry granulation, slugging, roll compaction, direct compression or any other technique known in the pharmaceutical art, wherein said formulation synergistically effects and extends the release of the API.

The wet granulation process comprises the following steps. (1) Dry blending the mixture of API, primary release modifying agent, secondary release modifying agent, auxiliary release modifying agent and other required pharmaceutically acceptable additives to make a uniform homogenous blend. (2) Wet granulating the uniform blend. (3) Diminuting the wet mass. (4) Drying and sizing the granules to an optimum size suitable for compression. (5) Blending the sized granules with the required pharmaceutically acceptable additives/lubricants. (6) Compressing the blended granules into tablets.

In another aspect of the invention, the homogenous blend of the active ingredient, the primary release modifying agent, the secondary release modifying agent, the auxiliary release modifying agent and the other required pharmaceutically acceptable additives is compacted into slugs or ribbons using a compression machine or roller compactor. The slugs or ribbons are reduced to the desired size, then lubricated and compressed into tablets.

In a further aspect of the invention, the homogenous blend of the active ingredient, the primary release modifying agent, the secondary release modifying agent, the auxiliary release modifying agent and the other required pharmaceutically acceptable additives is directly compressed into tablets. The pharmaceutical composition of the present invention can be prepared by any other technique known in the pharmaceutical art.

In the processes illustrated above, the pharmaceutical composition is preferably compressed into a tablet form, which has hardness in the order of 25 N to 350 N and more preferably 40 N to 300 N as determined by a Schleuniger hardness tester. The compressed tablets can be optionally coated with a coating, using the polymers or other coating agents which may or may not be intended or designed for the modification of drug release.

The following examples are provided to enable one skilled in the art to practice the invention and are merely illustrative of the invention. The examples should not be read as limiting the scope of the invention as defined in the claims.

### EXAMPLE 1

Nicotinic acid, which is a high soluble high dose API was mixed with high molecular weight polyethylene oxide (secondary release modifying agent), retrograded starch (auxiliary release modifying agent) and lactose monohydrate. The mixture was sifted through ASTM mesh no. 40, blended together in a blender to get a homogenous blend. The homogenous blend was granulated with water; the granules were dried in a fluid bed drier. The dried granules were reduced and sized to ASTM mesh no. 16 granules and then lubricated with talc and magnesium stearate.

TABLE 1-1

| | | Qty./ | |
|---|---|---|---|
| Sr. | | unit | % w/w of unit |
| No. | Ingredient | (mg) | dosage form |
| 1. | Nicotinic acid | 500.00 | 66.66 |
| 2. | Polyethylene Oxide | 170.00 | 22.66 |
| | (Mol. Wt.: 4,000,000) | | |
| 3. | Retrograded Starch | 40.00 | 5.33 |
| 4. | Lactose Monohydrate | 30.00 | 4.00 |
| 5. | Talc | 5.00 | 0.66 |
| 6. | Magnesium Stearate | 5.00 | 0.66 |
| 7. | Purified Water | q.s | q.s |

The drug release profile from the dosage form of the invention was studied in 900 ml of purified water in USP Dissolution Apparatus Type I at 100 RPM and the results are tabulated below. The results showed a controlled release of the drug from the dosage form of the Example 1. The drug release from the dosage form mentioned in example 1 was extended up to 24 h as shown in table 1-2.

TABLE 1-2

| | % Cumulative |
|---|---|
| Time (h) | Drug Released |
| 1 | 8.86 |
| 2 | 14.45 |
| 4 | 24.66 |
| 8 | 42.59 |
| 12 | 62.50 |
| 16 | 80.32 |
| 24 | 98.00 |

### EXAMPLE 3

Oxybutynin hydrochloride, which is a high soluble low dose API was mixed with high molecular weight polyethylene oxides (secondary release modifying agents), retrograded starch (auxiliary release modifying agent) and lactose monohydrate. The mixture was sifted through an ASTM mesh no. 40 and then blended together in a blender to get a homogenous blend. The homogenous blend was compacted into slugs or ribbons using a roller compactor. The slugs or ribbons are reduced to the desired size, then lubricated and compressed into tablets.

TABLE 2-1

| | | Qty./ | |
|---|---|---|---|
| Sr. | | unit | % w/w of unit |
| No. | Ingredient | (mg) | dosage form |
| 1. | Oxybutynin hydrochloride | 15.00 | 3.33 |
| 2. | Polyethylene Oxide | 15.00 | 3.33 |
| | (Mol. Wt.: 2,000,000) | | |
| 3. | Polyethylene Oxide | 300.00 | 66.66 |
| | (Mol. Wt.: 4,000,000) | | |
| 3. | Retrograded Starch | 45.00 | 10.00 |
| 4. | Lactose Monohydrate | 66.00 | 14.66 |
| 5. | Aerosil | 4.50 | 1.00 |
| 6. | Magnesium Stearate | 4.50 | 1.00 |

The drug release profile from the dosage form of the invention was studied in 500 ml of 0.1N Hydrochloric acid in USP Dissolution Apparatus Type II at 50 RPM and the results are tabulated below. The results showed a controlled release of the drug from the dosage form of the Example 2. The drug release from the dosage form mentioned in example 2 was extended up to 24 h as shown in Table 2-2.

TABLE 2-2

| | % Cumulative |
|---|---|
| Time (h) | Drug Released |
| 1 | 9 |
| 4 | 24 |
| 8 | 41 |
| 12 | 55 |
| 16 | 66 |
| 24 | 78 |

### EXAMPLE 3

Oxybutynin hydrochloride, which is a high soluble low dose API was mixed with high molecular weight polyethylene oxides (secondary release modifying agents), retrograded starch (auxiliary release modifying agent) and lactose monohydrate. The mixture was sifted through an ASTM mesh no. 40 and then blended together in a blender to get a homogenous blend. The homogenous blend was compacted into slugs or ribbons using a roller compactor. The slugs or ribbons are reduced to the desired size, then lubricated and compressed into tablets.

TABLE 3-1

| | | Qty./ | |
|---|---|---|---|
| Sr. | | unit | % w/w of unit |
| No. | Ingredient | (mg) | dosage form |
| 1. | Oxybutynin hydrochloride | 15.00 | 3.33 |
| 2. | Polyethylene Oxide | 15.00 | 3.33 |
| | (Mol. Wt.: 2,000,000) | | |
| 3. | Polyethylene Oxide | 240.00 | 53.33 |
| | (Mol. Wt.: 4,000,000) | | |
| 3. | Retrograded Starch | 45.00 | 10.00 |
| 4. | Lactose Monohydrate | 126.00 | 28.00 |
| 5. | Aerosil | 4.50 | 1.00 |
| 6. | Magnesium Stearate | 4.50 | 1.00 |

The drug release profile from the dosage form of the invention was studied in 500 ml of 0.1N Hydrochloric acid in USP Dissolution Apparatus Type II at 50 RPM and the results are tabulated below. The results showed a controlled release of the drug from the dosage form of the Example 3. The drug release from the dosage form mentioned in example 3 was extended up to 24 h as shown in Table 3-2.

TABLE 3-2

| | % Cumulative |
|---|---|
| Time (h) | Drug Released |
| 1 | 10 |
| 4 | 31 |
| 8 | 51 |
| 12 | 67 |
| 16 | 78 |
| 24 | 91 |

### EXAMPLE 3A

The tablets of Example 3 were coated with an aqueous dispersion of ethyl cellulose to a weight gain of 4 percent weight by weight of the dosage form. The aqueous dispersion of ethyl cellulose is commercially available as Surelease (E 719010) from Colorcon Pvt. Ltd.

The drug release profile from the coated dosage form of the invention was studied in 500 ml of 0.1N Hydrochloric acid in USP Dissolution Apparatus Type II at 50 RPM and the results are tabulated below. The results showed a controlled release of the drug from the dosage form of the Example 3A. The drug release from the dosage form mentioned in example 3A was extended up to 24 h. In the dosage form of example 3A, there was no drug release until the end of 4 hours and the drug release profile was extended up to 24 hours.

### EXAMPLE 4

Lercanidipine hydrochloride, which is a low soluble low dose API was mixed with low molecular weight polyethylene oxide (primary release modifying agent), high molecular weight polyethylene oxide (secondary release modifying agent), retrograded starch (auxiliary release modifying agent) and lactose monohydrate. The mixture was sifted through ASTM mesh no. 40 and then blended together in a blender to get a homogenous blend. The homogenous blend was compacted into slugs or ribbons using a roller compactor. The slugs or ribbons are reduced to the desired size, then lubricated and compressed into tablets.

TABLE 4-1

| | | Qty./ | % w/w of |
|---|---|---|---|
| Sr. | | unit | unit dosage |
| No. | Ingredient | (mg) | form |
| 1. | Lercanidipine hydrochloride | 20.00 | 13.33 |
| 2. | Polyethylene Oxide | 10.00 | 6.66 |
| | (Mol. Wt.: 2,00,000) | | |
| 3. | Polyethylene Oxide | 20.00 | 13.33 |
| | (Mol. Wt.: 2,000,000) | | |
| 3. | Retrograded Starch | 15.00 | 10.00 |
| 4. | Lactose Monohydrate | 81.50 | 54.33 |
| 5. | Aerosil | 0.80 | 0.533 |
| 6. | Magnesium Stearate | 2.70 | 1.80 |

The drug release profile from the dosage form of the invention was studied in 900 ml of 0.1N Hydrochloric acid with 1% sodium lauryl sulfate in USP Dissolution Apparatus Type II at 75 RPM and the results are tabulated below. The results showed a controlled release of the drug from the dosage form of the Example 4. The drug release from the dosage form mentioned in example 4 was extended up to 12 h as shown in table 4-2.

TABLE 4-2

| | % Cumulative |
|---|---|
| Time (h) | Drug Released |
| 1 | 13 |
| 2 | 26 |
| 4 | 58 |
| 6 | 90 |
| 8 | 92 |
| 12 | 92 |

### EXAMPLE 5

Lercanidipine hydrochloride, which is a low soluble low dose API was mixed with low molecular weight polyethylene oxide (primary release modifying agent), high molecular weight polyethylene oxide (secondary release modifying agent), retrograded starch (auxiliary release modifying agent) and lactose monohydrate. The mixture was sifted through ASTM mesh no. 40 and then blended together in a blender to get a homogenous blend. The homogenous blend was compacted into slugs or ribbons using a roller compactor. The slugs or ribbons are reduced to the desired size, then lubricated and compressed into tablets.

TABLE 5-1

| | | Qty./ | % w/w of |
|---|---|---|---|
| Sr. | | unit | unit dosage |
| No. | Ingredient | (mg) | form |
| 1. | Lercanidipine hydrochloride | 20.00 | 13.33 |
| 2. | Polyethylene Oxide | 5.00 | 3.33 |
| | (Mol. Wt.: 2,00,000) | | |
| 3. | Polyethylene Oxide | 10.00 | 6.66 |
| | (Mol. Wt.: 2,000,000) | | |
| 3. | Retrograded Starch | 10.00 | 6.66 |
| 4. | Lactose Monohydrate | 101.50 | 67.66 |
| 5. | Aerosil | 0.80 | 0.533 |
| 6. | Magnesium Stearate | 2.70 | 1.80 |

The drug release profile from the dosage form of the invention was studied in 900 ml of 0.1N Hydrochloric acid with 1% sodium lauryl sulfate in USP Dissolution Apparatus Type II at 75 RPM and the results are tabulated below. The results showed a controlled release of the drug from the dosage form of the Example 5. As shown in table 5-2, the drug release from the dosage form mentioned in example 5 was extended up to 12 h.

TABLE 5-2

| | % Cumulative |
|---|---|
| Time (h) | Drug Released |
| 1 | 22 |
| 2 | 37 |
| 4 | 72 |
| 6 | 79 |
| 8 | 80 |
| 12 | 81 |

### EXAMPLE 6

Clarithromycin, which is a low soluble high dose API, was mixed with a low molecular weight polyethylene oxide (primary release modifying agent) and/or high molecular weight polyethylene oxide (secondary release modifying agent), retrograded starch (auxiliary release modifying agent) and lactose monohydrate. The resulting mixture was sifted through ASTM mesh no. 40, blended together in a blender to get a homogenous blend. The homogenous blend was granulated with water; the granules were dried in a fluid bed drier. The dried granules were reduced and sized to ASTM mesh no. 16 granules and then lubricated with talc and magnesium stearate. The lubricated granules were compressed to tablets using the desired specific punches. The tablets were optionally coated with a polymer coating, using the polymers or coating agents not specifically designed for modification of drug release.

TABLE 6-1

| | | Qty./ | |
|---|---|---|---|
| Sr. | | unit | % w/w of unit |
| No. | Ingredient | (mg) | dosage form |
| 1. | Clarithromycin | 500.00 | 50.00 |
| 2. | Polyethylene Oxide | 150.00 | 15.00 |
| | (Mol. Wt.: 200,000) | | |
| 3. | Polyethylene Oxide (Mol. | 50.00 | 5.00 |
| | Wt.: 2,000,000) | | |
| 4. | Retrograded Starch | 150.00 | 15.00 |
| 5. | Lactose Monohydrate | 120.00 | 12.00 |
| 6. | Talc | 15.00 | 1.50 |
| 7. | Magnesium Stearate | 15.00 | 1.50 |
| 8. | Purified Water | q.s | q.s |

The drug release profile from the dosage form of the invention was studied in 900 ml of pH 5.0 acetate buffer in USP Dissolution Apparatus Type II at 50 RPM and the results are tabulated below. The results showed a controlled release of the drug from the dosage form of the Example 6. The drug release from the dosage form mentioned in example 6 was extended up to 12 h as shown in table 6-2.

TABLE 6-2

| | % Cumulative |
|---|---|
| Time (h) | Drug Released |
| 1 | 7 |
| 2 | 16 |
| 4 | 36 |
| 6 | 57 |
| 8 | 78 |
| 10 | 96 |
| 12 | 100 |

### EXAMPLE 7

TABLE 7-1

| | | Qty./ | |
|---|---|---|---|
| Sr. | | unit | % w/w of unit |
| No. | Ingredient | (mg) | dosage form |
| 1. | Clarithromycin | 500.0 | 50.00 |
| 2. | Polyethylene Oxide | 140.00 | 14.00 |
| | (Mol. Wt.: 200,000) | | |
| 3. | Polyethylene Oxide | 40.00 | 4.00 |
| | (Mol. Wt.: 2,000,000) | | |
| 4. | Retrograded Starch | 140.00 | 14.00 |
| 5. | Lactose Monohydrate | 135.00 | 13.50 |
| 6. | Talc | 30.00 | 3.00 |
| 7. | Magnesium Stearate | 15.00 | 1.50 |
| 8. | Purified Water | q.s | q.s |

The drug release profile from the dosage form of the invention was studied in 900 ml of pH 5.0 acetate buffer in USP Dissolution Apparatus Type II at 50 RPM and the results are tabulated below. The results showed a controlled release of the drug from the dosage form of the Example 7. The drug release from the dosage form mentioned in example 7 was extended up to 12 h, as shown in table 7-2.

TABLE 7-2

| | % Cumulative |
|---|---|
| Time (h) | Drug Released |
| 1 | 8 |
| 2 | 17 |
| 4 | 37 |
| 6 | 56 |
| 8 | 72 |
| 10 | 88 |
| 12 | 95 |

### EXAMPLE 8

TABLE 8-1

| | | Qty./ | |
|---|---|---|---|
| Sr. | | unit | % w/w of unit |
| No. | Ingredient | (mg) | dosage form |
| 1. | Clarithromycin | 500.00 | 50.00 |
| 2. | Polyethylene Oxide | 87.50 | 8.75 |
| | (Mol. Wt.: 200,000) | | |
| 3. | Polyethylene Oxide | 62.50 | 6.25 |
| | (Mol. Wt.: 2,000,000) | | |
| 4. | Retrograded Starch | 100.00 | 10.00 |
| 5. | Lactose Monohydrate | 205.00 | 20.50 |
| 6. | Talc | 30.00 | 3.00 |
| 7. | Magnesium Stearate | 15.00 | 1.50 |
| 8. | Purified Water | q.s | q.s |

The drug release profile from the dosage form of the invention was studied in 900 ml of pH 5.0 acetate buffer in USP Dissolution Apparatus Type II at 50 RPM and the results are tabulated below. The results showed a controlled release of the drug from the dosage form of the Example 8. The drug release from the dosage form mentioned in example 8 was extended up to 12 h, as shown in table 8-2.

TABLE 8-2

| | % Cumulative |
|---|---|
| Time (h) | Drug Released |
| 1 | 9 |
| 2 | 20 |
| 4 | 42 |
| 6 | 63 |
| 8 | 81 |
| 10 | 93 |
| 12 | 97 |

### EXAMPLE 9

TABLE 9-1

| | | Qty./ | |
|---|---|---|---|
| Sr. | | unit | % w/w of unit |
| No. | Ingredient | (mg) | dosage form |
| 1. | Clarithromycin | 500.00 | 50.00 |
| 2. | Polyethylene Oxide | 247.50 | 24.75 |
| | (Mol. Wt.: 200,000) | | |
| 3. | Polyethylene Oxide | 36.00 | 3.60 |
| | (Mol. Wt.: 2,000,000) | | |
| 4. | Retrograded Starch | 166.50 | 16.65 |
| 5. | Lactose Monohydrate | 5.00 | 0.50 |
| 6. | Talc | 30.00 | 3.00 |
| 7. | Magnesium Stearate | 15.00 | 1.50 |
| 8. | Purified Water | q.s | q.s |

The drug release profile from the dosage form of the invention was studied in 900 ml of pH 5.0 acetate buffer in USP Dissolution Apparatus Type II at 50 RPM and the results are tabulated below. The results showed a controlled release of the drug from the dosage form of the Example 9. The drug release from the dosage form mentioned in example 9 was extended up to 12 h, as shown in table 9-2.

TABLE 9-2

| | % Cumulative |
|---|---|
| Time (h) | Drug Released |
| 1 | 8 |
| 2 | 17 |
| 4 | 35 |
| 6 | 53 |
| 8 | 69 |
| 10 | 83 |
| 12 | 94 |

### EXAMPLE 10

TABLE 10-1

| | | Qty./ | |
|---|---|---|---|
| Sr. | | unit | % w/w of unit |
| No. | Ingredient | (mg) | dosage form |
| 1. | Clarithromycin | 500.00 | 50.00 |
| 2. | Polyethylene Oxide | 160.00 | 16.00 |
| | (Mol. Wt.: 200,000) | | |
| 3. | Polyethylene Oxide | 70.00 | 7.00 |
| | (Mol. Wt.: 2,000,000) | | |
| 4. | Retrograded Starch | 100.00 | 10.00 |
| 5. | Lactose Monohydrate | 125.00 | 12.50 |
| 6. | Talc | 30.00 | 3.00 |
| 7. | Magnesium Stearate | 15.00 | 1.50 |
| 8. | Purified Water | q.s | q.s |

The drug release profile from the dosage form of the invention was studied in 900 ml of pH 5.0 acetate buffer in USP Dissolution Apparatus Type II at 50 RPM and the results are tabulated below. The results showed a controlled release of the drug from the dosage form of the Example 10. The drug release from the dosage form mentioned in example 10 was extended up to 12 h, as shown in table 10-2.

TABLE 10-2

| | % Cumulative |
|---|---|
| Time (h) | Drug Released |
| 1 | 7 |
| 2 | 14 |
| 4 | 29 |
| 6 | 44 |
| 8 | 59 |
| 10 | 72 |
| 12 | 84 |

### EXAMPLE 11

TABLE 11-1

| | | Qty./ | |
|---|---|---|---|
| Sr. | | unit | % w/w of unit |
| No. | Ingredient | (mg) | dosage form |
| 1. | Clarithromycin | 500.00 | 50.00 |
| 2. | Polyethylene Oxide | 170.00 | 17.00 |
| | (Mol. Wt.: 200,000) | | |
| 3. | Polyethylene Oxide | 70.00 | 7.00 |
| | (Mol. Wt.: 2,000,000) | | |
| 4. | Retrograded Starch | 70.00 | 7.00 |
| 5. | Lactose Monohydrate | 145.00 | 14.50 |
| 6. | Talc | 30.00 | 3.00 |
| 7. | Magnesium Stearate | 15.00 | 1.50 |
| 8. | Purified Water | q.s | q.s |

The drug release profil from the dosage form of the invention was studied in 900 ml of pH 5.0 acetate buffer in USP Dissolution Apparatus Type II at 50 RPM and the results are tabulated below. The results showed a controlled release of the drug from the dosage form of the Example 11. The drug release from the dosage form mentioned in example 11 was extended up to 12 h, as shown in table 11-2.

TABLE 11-2

| | % Cumulative |
|---|---|
| Time (h) | Drug Released |
| 1 | 7 |
| 2 | 13 |
| 4 | 27 |
| 6 | 44 |
| 8 | 58 |
| 10 | 72 |
| 12 | 84 |

### EXAMPLE 12

TABLE 12-1

| | | Qty./ | |
|---|---|---|---|
| Sr. | | unit | % w/w of unit |
| No. | Ingredient | (mg) | dosage form |
| 1. | Clarithromycin | 500.00 | 50.00 |
| 2. | Polyethylene Oxide | 180.00 | 18.00 |
| | (Mol. Wt.: 200,000) | | |
| 3. | Polyethylene Oxide | 65.00 | 6.50 |
| | (Mol. Wt.: 2,000,000) | | |
| 4. | Retrograded Starch | 70.00 | 7.00 |
| 5. | Lactose Monohydrate | 140.00 | 14.00 |
| 6. | Talc | 30.00 | 3.00 |
| 7. | Magnesium Stearate | 15.00 | 1.50 |
| 8. | Purified Water | q.s | q.s |

The drug release profile from the dosage form of the invention was studied in 900 ml of pH 5.0 acetate buffer in USP Dissolution Apparatus Type II at 50 RPM and the results are tabulated below. The results showed a controlled release of the drug from the dosage form of the Example 12. The drug release from the dosage form mentioned in example 12 was extended up to 12 h, as shown in table 12-2.

TABLE 12-2

| | % Cumulative |
|---|---|
| Time (h) | Drug Released |
| 1 | 7 |
| 2 | 17 |
| 4 | 36 |
| 6 | 55 |
| 8 | 70 |
| 10 | 78 |
| 12 | 86 |

### EXAMPLE 13

TABLE 13-1

| | | Qty./ | |
|---|---|---|---|
| Sr. | | unit | % w/w of unit |
| No. | Ingredient | (mg) | dosage form |
| 1. | Clarithromycin | 500.00 | 50.00 |
| 2. | Polyethylene Oxide | 185.00 | 18.50 |
| | (Mol. Wt.: 200,000) | | |
| 3. | Polyethylene Oxide | 60.00 | 6.00 |
| | (Mol. Wt.: 2,000,000) | | |
| 4. | Retrograded Starch | 70.00 | 7.00 |
| 5. | Lactose Monohydrate | 140.00 | 14.00 |
| 6. | Talc | 30.00 | 3.00 |
| 7. | Magnesium Stearate | 15.00 | 1.50 |
| 8. | Purified Water | q.s | q.s |

The drug release profile from the dosage form of the invention was studied in 900 ml of pH 5.0 acetate buffer in USP Dissolution Apparatus Type II at 50 RPM and the results are tabulated below. The results showed a controlled release release from the dosage form of the Example 13. The drug release from the dosage form mentioned in example 13 was extended up to 12 h, as shown in table 13-2.

TABLE 13-2

| | % Cumulative |
|---|---|
| Time (h) | Drug Released |
| 1 | 9 |
| 2 | 18 |
| 4 | 35 |
| 6 | 52 |
| 8 | 66 |
| 10 | 79 |
| 12 | 89 |

### EXAMPLE 14

TABLE 14-1

| | | Qty./ | |
|---|---|---|---|
| Sr. | | unit | % w/w of unit |
| No. | Ingredient | (mg) | dosage form |
| 1. | Clarithromycin | 500.00 | 50.00 |
| 2. | Polyethylene Oxide | 240.00 | 22.00 |
| | (Mol. Wt.: 200,000) | | |
| 3. | Retrograded Starch | 120.00 | 10.00 |
| 4. | Lactose Monohydrate | 135.00 | 13.50 |
| 5. | Talc | 30.00 | 3.00 |
| 6. | Magnesium Stearate | 15.00 | 1.50 |
| 7. | Purified Water | q.s | q.s |

The drug release profile from the dosage form of the invention was studied in 900 ml of pH 5.0 acetate buffer in USP Dissolution Apparatus Type II at 50 RPM and the results are tabulated below. The results showed a controlled release of the drug from the dosage form of the Example 14. The drug release from the dosage form mentioned in example 14 was extended up to 12 h, as shown in table 14-2.

TABLE 14-2

| | % Cumulative |
|---|---|
| Time (h) | Drug Released |
| 1 | 20 |
| 2 | 35 |
| 4 | 65 |
| 6 | 80 |
| 8 | 90 |
| 10 | 95 |
| 12 | 97 |

### EXAMPLE 15

TABLE 15-1

| | | Qty./ | |
|---|---|---|---|
| Sr. | | unit | % w/w of unit |
| No. | Ingredient | (mg) | dosage form |
| 1. | Clarithromycin | 500.00 | 50.00 |
| 2. | Polyethylene Oxide | 55.00 | 7.50 |
| | (Mol. Wt.: 2,000,000) | | |
| 3. | Retrograded Starch | 120.00 | 12.00 |
| 4. | Lactose Monohydrate | 260.00 | 26.00 |
| 5. | Talc | 30.00 | 3.00 |
| 6. | Magnesium Stearate | 15.00 | 1.50 |
| 7. | Purified Water | q.s | q.s |

The dosage release profile from the dosage form of the invention was studied in 900 ml of pH 5.0 acetate buffer in USP Dissolution Apparatus Type II at 50 RPM and the results are tabulated below. The results showed a controlled release of the drug from the dosage form of the Example 15. The drug release from the dosage form mentioned in example 15 was extended up to 12 h, as shown in table 15-2.

TABLE 15-2

| | % Cumulative |
|---|---|
| Time (h) | Drug Released |
| 1 | 10 |
| 2 | 20 |
| 4 | 35 |
| 6 | 40 |
| 8 | 50 |
| 10 | 60 |
| 12 | 80 |

### EXAMPLE 16

Alprazolam which is a low soluble low dose API was mixed with low molecular weight polyethylene oxide (primary release modifying agent), retrograded starch (auxiliary release modifying agent), lactose monohydrate and magnesium stearate. The mixture was sifted through ASTM mesh no. 40 and then blended together in a blender to get a homogenous blend. The homogenous blend was compressed into tablets.

TABLE 16-1

| | | Qty./ | % w/w of |
|---|---|---|---|
| Sr. | | unit | unit dosage |
| No. | Ingredient | (mg) | form |
| 1. | Alprazolam | 0.50 | 0.50 |
| 2. | Polyethylene Oxide | 5.00 | 5.00 |
| | (Mol. Wt.: 2,00,000) | | |
| 3. | Retrograded Starch | 65.00 | 65.00 |
| 4. | Lactose Monohydrate | 29.00 | 29.00 |
| 5. | Magnesium Stearate | 0.50 | 0.50 |

The drug release profil from the dosage form of the invention was studied in 500 ml of pH 6.8 phosphate buffer in USP Dissolution Apparatus Type I at 100 RPM and the results are tabulated below. The results showed a controlled release of the drug from the dosage form of the Example 16. As shown in table 16-2, the drug release from the dosage form mentioned in example 16 was extended up to 12 h, as shown in table 16-2.

TABLE 16-2

| | % Cumulative |
|---|---|
| Time (h) | Drug Released |
| 1 | 11 |
| 2 | 29 |
| 4 | 52 |
| 6 | 70 |
| 8 | 84 |
| 12 | 96 |

The pharmaceutical compostions of the present invention mentioned in the above examples shows a predictable and predetermined controlled drug release profile devoid of dose dumping and the drug release was almost complete over an extended duration of about 12 to 24 hours depending on the requirement for a particular API. The difference between the drug release from the individual unit dosage forms is insignificant.

One skilled in the art would recognize that the specifically recited drugs Nicotinic Acid, Oxybutynin hydrochloride, Lercanidipine hydrochloride, Clarithromycin, and Alprazolam, their respective dosing and solubility ranges are only representative examples of the present invention and are not meant to limit the scope of the invention to the specific drugs or drug profiles recited herein. One of skill in the art would also recognize that a wide range of drug compositions would be determined from the scope of the invention, as well as their desired profiles.

Pharmacokinetic Study: The pharmaceutical composition of the invention as described in Example 13 was subjected to a bioavailability study against the commercially available Clarithromycin 500 mg Extended Release Tablets (Biaxin XL Filmtab, 500 mg).

An open label, randomized, two-treatment, two-period, two-sequence, single dose, crossover oral bioavailability study in healthy, adult male subjects was conducted on the composition of Example 13 of the present invention administered once daily with Biaxin XL Filmtab, 500 mg tablet of Abbott Laboratories.

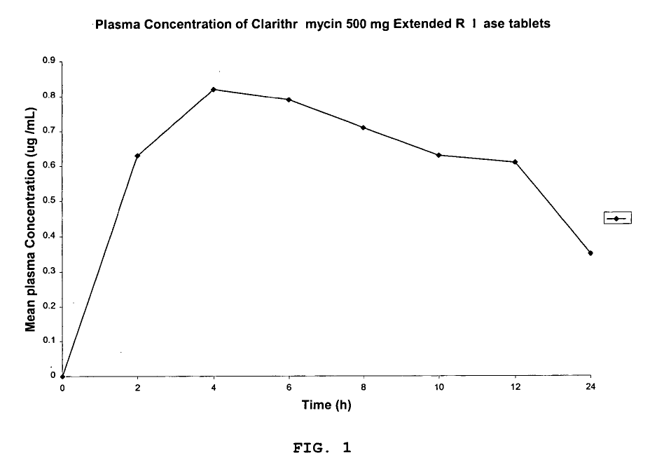

The mean plasma concentration-time profiles for the single-dose study are illustrated in FIG. 1.

The log transformed pharmacokinetic parameters for the two clarithromycin extended release compositions are tabulated in Table 13-3.

TABLE 13-3

| Formula Composition | | |
|---|---|---|
| Comparison | Cmax (μg./ml) | AUC₀₋ₜ (μg.hr./ml) |
| Biaxin XL Filmtab, | 1.01 | 13.61 |
| 500 mg | | |
| (Reference) | | |
| Clarithromycin | 1.06 | 13.72 |
| Extended Release | | |
| Composition of | | |
| Example 8 | | |
| (Test) | | |

Point estimates of the relative bioavailability and 90% confidence intervals from the log transformed values are tabulated in Table 13-4.

TABLE 13-4

| Formula Composition | | |
|---|---|---|
| Comparison | Cmax (μg./ml) | AUC₀₋ₜ (μg.hr./ml) |
| Ratio (%) | 104.19 | 100.75 |
| Test/Reference | | |
| 90% Confidence | 84.41-128.61 | 81.63-124.36 |
| Interval | | |

Although the invention herein has been described with reference to particular embodiments, it is to be understood that these embodiments are merely illustrative of the principles and applications of the present invention. It is therefore to be understood that numerous modifications may be made- to the illustrative embodiments and that other arrangements may be devised without departing from the spirit and scope of the present invention as defined by the above descriptions, examples and the claims.

All patent and non-patent publications cited in this specification are indicative of the level of skill of those skilled in the art to which this invention pertains. All these publications and patent applications are herein incorporated by reference to the same extent as if each individual publication or patent application was specifically and individually indicated to be incorporated herein by reference.

1. A solid oral controlled release pharmaceutical composition for administration to a subject in need thereof comprising:
(a) a therapeutically effective amount of a pharmaceutically active ingredient; and
(b) a controlled release modifying complex wherein said complex comprises:
(i) a primary release modifying agent;
(ii) a secondary release modifying agent; and
(iii) an auxiliary release modifying agent wherein said primary, secondary and auxiliary release modifying agents are present in amounts that synergistically extend the release of the pharmaceutically active ingredient.

2. A solid oral controlled release pharmaceutical composition for administration to a subject in need thereof comprising:
(a) a therapeutically effective amount of a pharmaceutically active ingredient; and
(b) a controlled release modifying complex wherein said complex comprises:
(i) a primary release modifying agent; or
(ii) a secondary release modifying agent; and
(iii) an auxiliary release modifying agent wherein said primary, secondary and auxiliary release modifying agents are present in amounts that synergistically extend the release of the pharmaceutically active ingredient.

3. A solid oral controlled release pharmaceutical composition for administration to a subject in need thereof comprising:
(a) a therapeutically effective amount of a pharmaceutically active ingredient; and
(b) a controlled release modifying complex
wherein said complex comprises:
(i) a primary release modifying agent selected from low molecular weight hydrophilic polymers;
(ii) a secondary release modifying agent selected from high molecular weight hydrophilic polymers; and
(iii) an auxiliary release modifying agent selected from the starch derivatives
wherein said primary, secondary and auxiliary release modifying agents are present in amounts that synergistically extend the release of the pharmaceutically active ingredient.

4. A solid oral controlled release pharmaceutical composition for administration to a subject in need thereof comprising:
(a) a therapeutically effective amount of a pharmaceutically active ingredient; and
(b) a controlled release modifying complex
wherein said complex comprises:
(i) a primary release modifying agent selected from low molecular weight hydrophilic polymers; or
(ii) a secondary release modifying agent selected from high molecular weight hydrophilic polymers; and
(iii) an auxiliary release modifying agent selected from the starch derivatives
wherein said primary, secondary and auxiliary release modifying agents are present in amounts that synergistically extend the release of the pharmaceutically active ingredient.

5. The pharmaceutical composition of claim 1 wherein the dosage form is a tablet, caplet or capsule.

6. The pharmaceutical composition of claim 1, wherein the active pharmaceutical ingredient is a macrolide or azide antibiotic or a derivative thereof.

7. The pharmaceutical composition of claim 6, wherein the macrolide is an erythromycin derivative or its pharmaceutically acceptable hydrates, salts or esters.

8. The pharmaceutical composition of claim 6, wherein the azide is azithromycin, or its pharmaceutically acceptable hydrates, salts or esters.

9. The pharmaceutical composition of claim 7, wherein the erythromycin derivative is selected from the group consisting of clarithromycin, josamycin, midecamycin, kitamycin, roxithromycin, rokitamycin, oleandomycin, miocamycin, flurithromycin, and rosaramicin, and their pharmaceutically acceptable hydrates, salts and esters.

10. The pharmaceutical composition of claim 1, wherein the active pharmaceutical ingredient is a penicillin antibiotic or a derivative thereof.

11. The pharmaceutical composition of claim 10, wherein the penicillin derivative is selected from the group consisting of Amoxicillin, Ampicillin, Ampicillin Sodium, Apalcillin, Aspoxicillin, Azlocillin, Aztreonam, Bacampicillin, Cabenicillin, Carfecillin, Carindacillin, Ciclacillin, Cloxacillin, Dicloxacillin, and their pharmaceutically acceptable hydrates, salts and esters.

12. The pharmaceutical composition of claim 1, wherein the active pharmaceutical ingredient is a cephalosporin antibiotic or a derivative thereof.

13. The pharmaceutical composition of claim 12, wherein the cephalosporin derivative is selected from the group consisting of Cefacetrile, Cefadroxil, Cefaloridine, Cefalothin Sodium, Cefapirin, Cefazaflur, Cefazedone, Cefazolin, Cefradine, Ceftezole, Cefsulodin Sodium, Cefamandole, Cefonicid, Cefoperazone, Cefuroxime, Cefuzonam, Cefbuperazone, Cefoxitin, Cefminox, Cefinetazole, Cefotetan, Loracarbef, Cefinenoxime, Cefodizime Sodium, Cefotaxime, Cefpimizole, Cefpiramide, Ceftazidime, Ceftiolene, Ceftizoxime, Ceftriaxone, Cefepime, Cefetecol, Cefpirome, Cefquinome, Cefalosporin C, Cefozopran Hydrochloride, Cefaclor, Cefadroxil, Cefalexin, Cefaloglycine, Cefatrizine, Cefdinir, Cefetamet Pivoxil, Cefixime, Ceforamide, Cefotiam, Cefpodoxime, Cefpodoxime Proxetil, Cefprozil, Cefradine, Cefroxadine, Cefteram Pivoxil, Ceftibuten, Cefuroxime Axetil, and their pharmaceutically acceptable hydrates, salts and esters.

14. The pharmaceutical composition of claim 1, wherein the active pharmaceutical ingredient is a quinolone antibiotic or a derivative thereof.

15. The pharmaceutical composition of claim 14, wherein the quinolone derivative is selected from the group consisting of Nalidixic Acid, Cinoxacin, Oxolinic Acid, Flumequine, Pipemidic Acid, Rosoxacin, Norfloxacin, Lomefloxacin, Ofloxacin, Enrofloxacin, Ciprofloxacin, Enoxacin, Amifloxacin, Fleroxacin, Gatifloxacin, Gemifloxacin, Pefloxacin, Rufloxacin, Sparfloxacin, Temafloxacin, Tosufloxacin, Grepafloxacin, Levofloxacin, Moxifloxacin, Trovafloxacin, and their pharmaceutically acceptable hydrates, salts and esters.

16. The pharmaceutical composition of claim 1, wherein the active pharmaceutical ingredient is a high soluble high dose API or a derivative thereof.

17. The pharmaceutical composition of claim 16, wherein the high soluble high dose API or a derivative is selected from the group consisting of Acebutolol hydrochloride, Amantadine hydrochloride, Aminocaproic acid, Aminophylline, Amodiaquine hydrochloride, Ascorbic acid, Bupropion hydrochloride, Carbenoxolone sodium, Cefuroxime sodium, Chloroquine phosphate, Chloroquine sulphate, Chlorpromazine hydrochloride, Ciprofloxacin hydrochloride, Cloxacillin sodium, Cycloserine, Diltiazem hydrochloride, Diethyl carbamazine citrate, Doxycycline hydrochloride, Ethosuximide, Ferrous gluconate, Isoniazid, Levamisole hydrochloride, Levetiracetam, Lincomycin hydrochloride, Mebeverine hydrochloride, Mepyramine maleate, Metformin hydrochloride, Metoprolol tartrate, Metoprolol succinate, Niacin, Nicotinamide, Nicotinic acid, Oxprenolol hydrochloride, Oxytetracycline hydrochloride, Penicillamine, Pentobarbitone sodium, Phenoxy Methyl Penicillin K, Phenyloin sodium, piperazine adipate, Potassium chloride, Procainamide hydrochloride, Propranolol hydrochloride, Pseudoephedrine hydrochloride, Quinalbarbitone sodium, Quinine bisulphate, Ranitidine hydrochloride, Sodium amino salicylate, Sodium fusidate, sodium valproate, Streptomycin sulphate, Tetracycline hydrochloride, Topiramate, Troxidone, Verapamil hydrochloride and the like and their pharmaceutically acceptable salts, ester and hydrates.

18. The pharmaceutical composition of claim 17, wherein the active ingredient is Nicotinic acid.

19. The pharmaceutical composition of claim 1, wherein the active pharmaceutical ingredient is a high soluble low dose API or a derivative thereof.

20. The pharmaceutical composition of claim 19, wherein the high soluble low dose API or a derivative is selected from the group consisting of Amitriptylline hydrochloride, captopril, Clonidine hydrochloride, Colchicin, Cyclophosphamide, Diphenhydramine hydrochloride, Dothiepen hydrochloride, Doxepin hydrochloride Ephedrine hydrochloride, Ergometrine maleate, Ergometrine tartrate, Fenfluramine hydrochloride, Folic acid, Glyceryl trinitrate, Hyoscine hydrobromide, Hyoscine buytlbromide, Imipramine hydrochloride, Isoprenaline sulphate, Isosorbide dinitrate, Isosorbide-5-mononitrate, Levocetrizine hydrochloride, Methadone hydrochloride, Methdilazine hydrochloride, Metoclopramide hydrochloride, Neostigmine bromide, Oxybutynin hydrochloride, Perindopril erbumine, Pethidine hydrochloride, Phenformin hydrochloride, Pheniramine maleate, Phenobarbitone sodium, Primaquine phosphate, Promethazine hydrochloride, Propantheline bromide, Propranolol hydrochloride, Pyridoxine hydrochloride, Salbutamol sulphate, Terbutaline sulphate, Thiamine hydrochloride, Timolol maleate, Tizanidine hydrochloride, Trifluoperazine hydrochloride, Triflupromazine hydrochloride, Triprolidine hydrochloride, Warfarin sodium and the like and their pharmaceutically acceptable salts, ester and hydrates.

21. The pharmaceutical composition of claim 20, wherein the active ingredient is Oxybutynin hydrochloride.

22. The pharmaceutical composition of claim 1, wherein the active pharmaceutical ingredient is a low soluble high dose API or a derivative thereof.

23. The pharmaceutical composition of claim 22, wherein the low soluble high dose API or a derivative is selected from the group consisting of Acetazolamide, Allopurinol, Atenolol, Carbamazepine, Cefadroxil, Cephalexin, Chloramphenicol, Cefuroxime Axetil, Chlorthalidone, Cilastozol, Cimetidine, Clarithromycin, Clofazemine, Dapsone, Diclofenac sodium, Diiodohydroxy quinolone, Diloxamide furoate, Disulfiram, Erythromycin, Erythromycin estolate, Erythromycin stearate, Ethacrynic cid, Ethionamide, Ethopropazine hydrochloride, Ferrous fumarate, Fluconazole, Flurbiprofen, Furazolidone, Griseofulvin, Hydrochlorthiazide, Ibuprofen, Itraconazole, Ketoconazole, Ketoprofen, Labetalol hydrochloride, Levodopa, Linezolid, Lithium carbonate, Magaldrate, Mebendazole, Mefenamic acid, Megestrol acetate, Mercaptopurine, Mesalamimne, Nalidixic acid, Nateglinide, Niclosamide, Nitrofurantoin, Norfloxacin, Oxcarbazepine, Oxyphenbutazone, Paracetamol, Phenindione, Phenobarbitone, Phenylbutazone, Phenylsulphathiazole, piperazine phosphate, Proguanil hydrochloride, Promethazine theoclate, Propylthiouracil, Quinidine sulphate, Quinine sulphate, Quinidochlor, Rifampicin, Simvastatin, Spironolactone, Succinylsulphathiazole, Sulphadiazine, Sulphadimethoxine, Sulphadimidine, Sulphafurazole, Sulphaphenazole, Thiabendazole, Timidazole, Tolbutamide, Triamterene, Sulphamethoxazole and the like and their pharmaceutically acceptable salts, ester and hydrates.

24. The pharmaceutical composition of claim 23, wherein the active ingredient is Clarithromycin.

25. The pharmaceutical composition of claim 1, wherein the active pharmaceutical ingredient is a low soluble low dose API or a derivative thereof.

26. The pharmaceutical composition of claim 25, wherein the low soluble low dose API or a derivative is selected from the group consisting of Alprazolam, Amiloride hydrochloride, Astemizole, Atorvastatin, Benzhexol hydrochloride, Betamethasone, Bromhexine hydrochloride, Bromocryptine mesylate, Buprenorphine hydrochloride, carbimazole, Chlordiazepoxide, Citalopram, Cortisone acetate, cyproheptadine hydrochloride, diazepam, Desloratadine, Dexamethasone hydrochloride, Dextromethorphan hydrochloride, dicyclomine hydrochloride, Dienoestrol, Digitoxin, Digoxin, Domperidone, Dydrogesterone, enalapril maleate, Esomeprazole, Ethinyloestradiol, Ethyloestrenol, Fludrocortisone acetate, Frusemide, glibenclamide, Glimepiride, haloperidol, Indomethacin, Isoxsuprine hydrochloride, Lanatoside C, Lercanidipine hydrochloride, Levonorgesrtel, Lomustine, Loratadine, Isoxuprine hydrochloride, methotrexate, Mecizine hydrochloride, Melphalan, Methotrexate, Methylergometrine maleate, Methylprednisolone, Mianserin hydrochloride, Mosapride, Nicoumalone, Nifedipine, Nitrazepam, Norethisterone, nortriptyline hydrochloride, Omeprazole, Ormeloxifene hydrochloride, Pentazocine hydrochloride, Phenindamine tartrate, piroxicam, prazosin hydrochloride, Prednisolone, Prednisone, Prochlorperazine maleate, Repaglinide, Riboflavinee, Rosuvastatin, Stilbostrol, Tamoxifen citrate, Thyroxine sodium, triamcinilone, Trimethoprim, Valdecoxib, Zolpidem tartrate and the like and their pharmaceutically acceptable salts, ester and hydrates.

27. The pharmaceutical composition of claim 26, wherein the active ingredient is Alprazolam.

28. The pharmaceutical composition of claim 26, wherein the active ingredient is Lercanidipine hydrochloride.

29. The pharmaceutical composition of claim 1, wherein the primary release modifying agent is a low molecular weight polyethylene oxide.

30. The pharmaceutical composition of claim 1, wherein the low molecular weight polyethylene oxide has a molecular weight of at least about 100,000.

31. The pharmaceutical composition of claim 1 wherein the low molecular weight polyethylene oxide has a molecular weight ranging from 100,000 to 900,000.

32. The pharmaceutical composition of claim 1, wherein the secondary release modifying agent is a high molecular weight polyethylene oxide.

33. The pharmaceutical composition of claim 1, wherein the high molecular weight polyethylene oxide has a molecular weight of at least about 1,000,000.

34. The pharmaceutical composition of claim 1, wherein the high molecular weight polyethylene oxide has a molecular weight ranging from 1,000,000 to 9,000,000.

35. The pharmaceutical composition of claim 1, wherein the auxiliary release modifying agent is a starch derivative selected from pregelatinized starch, partially pregelatinized starch, retrograded starch, or a combination thereof.

36. The pharmaceutical composition of claim 1, wherein the auxiliary release modifying agent is a retrograded starch.

37. The pharmaceutical composition of claim 1, wherein said composition further comprises at least one pharmaceutically acceptable additive selected from diluents, fillers, binders, glidants, and lubricants.

38. The pharmaceutical composition of claim 1, wherein said composition further comprises an optional coating which is designed for the modification of drug release.

39. The pharmaceutical composition of claim 38, wherein the coating composition is selected from the group consisting of cellulose ethers such as ethyl cellulose, hydroxypropyl methyl cellulose, hydroxypropyl cellulose, and others such as polyvinyl alcohol, polyvinyl pyrrolidone, methacrylic acid derivatives, resins, clays, long chain hydrocarbons, long chain carboxylic acids, long chain carboxylic acid esters, long chain alcohols and the like and mixtures thereof.

40. The pharmaceutical composition of claim 1, wherein said composition further comprises an optional coating which is not designed for the modification of drug release.

41. The pharmaceutical composition of claim 40, wherein the coating composition is selected from the group consisting of cellulose ethers such as ethyl cellulose, hydroxypropyl methyl cellulose, hydroxypropyl cellulose, and others such as polyvinyl alcohol, polyvinyl pyrrolidone, methacrylic acid derivatives, resins, clays, long chain hydrocarbons, long chain carboxylic acids, long chain carboxylic acid esters, long chain alcohols and the like and mixtures thereof.

42. The pharmaceutical composition of claim 1, wherein said composition comprises from about 0.1 percent weight to about 90 percent weight of the active pharmaceutical ingredient.

43. The pharmaceutical composition of claim 42, wherein said composition comprises from about 0.1 percent weight to about 80 percent weight of the active pharmaceutical ingredient.

44. The pharmaceutical composition of claim 1, wherein the composition comprises from about 1 percent weight to about 90 percent weight of the release modifying complex.

45. The pharmaceutical composition of claim 44, wherein the composition comprises from about 5 percent weight to about 85 percent weight of the release modifying complex.

46. The pharmaceutical composition of claim 45, wherein the composition comprises from about 10 percent weight to about 80 percent weight of the release modifying complex.

47. The pharmaceutical composition of claim 1, wherein the primary release modifying agent comprises from about 1 percent weight to about 90 percent weight of the release modifying complex.

48. The pharmaceutical composition of claim 47, wherein the primary release modifying agent comprises from about 5 percent weight to about 80 percent weight of the release modifying complex.

49. The pharmaceutical composition of claim 48, wherein the primary release modifying agent comprises from about 5 percent weight to about 70 percent weight of the release modifying complex.

50. The pharmaceutical composition of claim 1, wherein the secondary release modifying agent comprises from about 1 percent weight to about 95 percent weight of the release modifying complex.

51. The pharmaceutical composition of claim 50, wherein the secondary release modifying agent comprises from about 5 percent weight to about 90 percent weight of the release modifying complex.

52. The pharmaceutical composition of claim 1, wherein the auxiliary release modifying agent comprises from about 1 percent weight to about 95 percent weight of the release modifying complex.

53. The pharmaceutical composition of claim 52, wherein the auxiliary release modifying agent comprises from about 5 percent weight to about 95 percent weight of the release modifying complex.

54. The pharmaceutical composition of claim 53, wherein the auxiliary release modifying agent comprises from about 10 percent weight to about 95 percent weight of the release modifying complex.

55. A controlled release pharmaceutical composition comprising a pharmaceutically active ingredient and a release modifying complex, wherein said release modifying complex comprises a synergistic combination of (a) a primary release modifying agent selected from a low molecular weight polyethylene oxide and (b) an auxiliary release modifying agent selected from a starch derivative, and said primary release modifying agent and auxiliary release modifying agent are present in synergistic effective amounts to extend the release of the active pharmaceutical ingredient.

56. The pharmaceutical composition of claim 55, wherein the active pharmaceutical ingredient is a macrolide or azide antibiotic or a derivative thereof.

57. The pharmaceutical composition of claim 56, wherein the macrolide is an erythromycin derivative or its pharmaceutically acceptable hydrates, salts or esters.

58. The pharmaceutical composition of claim 56, wherein the azide is azithromycin, or its pharmaceutically acceptable hydrates, salts or esters.

59. The pharmaceutical composition of claim 57, wherein the erythromycin derivative is selected from the group consisting of clarithromycin, josamycin, midecamycin, kitamycin, roxithromycin, rokitamycin, oleandomycin, miocamycin, flurithromycin, and rosaramicin, and their pharmaceutically acceptable hydrates, salts and esters.

60. The pharmaceutical composition of claim 55, wherein the active pharmaceutical ingredient is a penicillin antibiotic or a derivative thereof.

61. The pharmaceutical composition of claim 60, wherein the penicillin derivative is selected from the group consisting of Amoxicillin, Ampicillin, Ampicillin Sodium, Apalcillin, Aspoxicillin, Azlocillin, Aztreonam, Bacampicillin, Cabenicillin, Carfecillin, Carindacillin, Ciclacillin, Cloxacillin, Dicloxacillin, and their pharmaceutically acceptable hydrates, salts and esters.

62. The pharmaceutical composition of claim 55, wherein the active pharmaceutical ingredient is a cephalosporin antibiotic or a derivative thereof.

63. The pharmaceutical composition of claim 62, wherein the cephalosporin derivative is selected from the group consisting of Cefacetrile, Cefadroxil, Cefaloridine, Cefalothin Sodium, Cefapirin, Cefazaflur, Cefazedone, Cefazolin, Cefradine, Ceftezole, Cefsulodin Sodium, Cefamandole, Cefonicid, Cefoperazone, Cefuroxime, Cefuzonam, Cefbuperazone, Cefoxitin, Cefminox, Cefinetazole, Cefotetan, Loracarbef, Cefinenoxime, Cefodizime Sodium, Cefotaxime, Cefpimizole, Cefpiramide, Ceftazidime, Ceftiolene, Ceftizoxime, Ceftriaxone, Cefepime, Cefetecol, Cefpirome, Cefquinome, Cefalosporin C, Cefozopran Hydrochloride, Cefaclor, Cefadroxil, Cefalexin, Cefaloglycine, Cefatrizine, Cefdinir, Cefetamet Pivoxil, Cefixime, Ceforamide, Cefotiam, Cefpodoxime, Cefpodoxime Proxetil, Cefprozil, Cefradine, Cefroxadine, Cefteram Pivoxil, Ceftibuten, Cefuroxime Axetil, and their pharmaceutically acceptable hydrates, salts and esters.

64. The pharmaceutical composition of claim 55, wherein the active pharmaceutical ingredient is a quinolone antibiotic or a derivative thereof.

65. The pharmaceutical composition of claim 64, wherein the quinolone derivative is selected from the group consisting of Nalidixic Acid, Cinoxacin, Oxolinic Acid, Flumequine, Pipemidic Acid, Rosoxacin, Norfloxacin, Lomefloxacin, Ofloxacin, Enrofloxacin, Ciprofloxacin, Enoxacin, Amifloxacin, Fleroxacin, Gatifloxacin, Gemifloxacin, Pefloxacin, Rufloxacin, Sparfloxacin, Temafloxacin, Tosufloxacin, Grepafloxacin, Levofloxacin, Moxifloxacin, Trovafloxacin, and their pharmaceutically acceptable hydrates, salts and esters.

66. The pharmaceutical composition of claim 55, wherein the active pharmaceutical ingredient is a high soluble high dose API or a derivative thereof.

67. The pharmaceutical composition of claim 66, wherein the high soluble high dose API or a derivative is selected from the group consisting of Acebutolol hydrochloride, Amantadine hydrochloride, Aminocaproic acid, Aminophylline, Amodiaquine hydrochloride, Ascorbic acid, Bupropion hydrochloride, Carbenoxolone sodium, Cefuroxime sodium, Chloroquine phosphate, Chloroquine sulphate, Chlorpromazine hydrochloride, Ciprofloxacin hydrochloride, Cloxacillin sodium, Cycloserine, Diltiazem hydrochloride, Diethyl carbamazine citrate, Doxycycline hydrochloride, Ethosuximide, Ferrous gluconate, Isoniazid, Levamisole hydrochloride, Levetiracetam, Lincomycin hydrochloride, Mebeverine hydrochloride, Mepyramine maleate, Metformin hydrochloride, Metoprolol tartrate, Metoprolol succinate, Niacin, Nicotinamide, Nicotinic acid, Oxprenolol hydrochloride, Oxytetracycline hydrochloride, Penicillamine, Pentobarbitone sodium, Phenoxy Methyl Penicillin K, Phenyloin sodium, piperazine adipate, Potassium chloride, Procainamide hydrochloride, Propranolol hydrochloride, Pseudoephedrine hydrochloride, Quinalbarbitone sodium, Quinine bisulphate, Ranitidine hydrochloride, Sodium amino salicylate, Sodium fusidate, sodium valproate, Streptomycin sulphate, Tetracycline hydrochloride, Topiramate, Troxidone, Verapamil hydrochloride and the like and their pharmaceutically acceptable salts, ester and hydrates.

68. The pharmaceutical composition of claim 67, wherein the active ingredient is Nicotinic acid.

69. The pharmaceutical composition of claim 55, wherein the active pharmaceutical ingredient is a high soluble low dose API or a derivative thereof.

70. The pharmaceutical composition of claim 69, wherein the high soluble low dose API or a derivative is selected from the group consisting of Amitriptylline hydrochloride, captopril, Clonidine hydrochloride, Colchicin, Cyclophosphamide, Diphenhydramine hydrochloride, Dothiepen hydrochloride, Doxepin hydrochloride Ephedrine hydrochloride, Ergometrine maleate, Ergometrine tartrate, Fenfluramine hydrochloride, Folic acid, Glyceryl trinitrate, Hyoscine hydrobromide, Hyoscine buytlbromide, Imipramine hydrochloride, Isoprenaline sulphate, Isosorbide dinitrate, Isosorbide-5-mononitrate, Levocetrizine hydrochloride, Methadone hydrochloride, Methdilazine hydrochloride, Metoclopramide hydrochloride, Neostigmine bromide, Oxybutynin hydrochloride, Perindopril erbumine, Pethidine hydrochloride, Phenformin hydrochloride, Pheniramine maleate, Phenobarbitone sodium, Primaquine phosphate, Promethazine hydrochloride, Propantheline bromide, Propranolol hydrochloride, Pyridoxine hydrochloride, Salbutamol sulphate, Terbutaline sulphate, Thiamine hydrochloride, Timolol maleate, Tizanidine hydrochloride, Trifluoperazine hydrochloride, Triflupromazine hydrochloride, Triprolidine hydrochloride, Warfarin sodium and the like and their pharmaceutically acceptable salts, ester and hydrates.

71. The pharmaceutical composition of claim 70, wherein the active ingredient is Oxybutynin hydrochloride.

72. The pharmaceutical composition of claim 55, wherein the active pharmaceutical ingredient is a low soluble high dose API or a derivative thereof.

73. The pharmaceutical composition of claim 72, wherein the low soluble high dose API or a derivative is selected from the group consisting of Acetazolamide, Allopurinol, Atenolol, Carbamazepine, Cefadroxil, Cephalexin, Chloramphenicol, Cefuroxime Axetil, Chlorthalidone, Cilastozol, Cimetidine, Clarithromycin, Clofazemine, Dapsone, Diclofenac sodium, Diiodohydroxy quinolone, Diloxamide furoate, Disulfiram, Erythromycin, Erythromycin estolate, Erythromycin stearate, Ethacrynic cid, Ethionamide, Ethopropazine hydrochloride, Ferrous fumarate, Fluconazole, Flurbiprofen, Furazolidone, Griseofulvin, Hydrochlorthiazide, Ibuprofen, Itraconazole, Ketoconazole, Ketoprofen, Labetalol hydrochloride, Levodopa, Linezolid, Lithium carbonate, Magaldrate, Mebendazole, Mefenamic acid, Megestrol acetate, Mercaptopurine, Mesalamimne, Nalidixic acid, Nateglinide, Niclosamide, Nitrofurantoin, Norfloxacin, Oxcarbazepine, Oxyphenbutazone, Paracetamol, Phenindione, Phenobarbitone, Phenylbutazone, Phenylsulphathiazole, piperazine phosphate, Proguanil hydrochloride, Promethazine theoclate, Propylthiouracil, Quinidine sulphate, Quinine sulphate, Quinidochlor, Rifampicin, Simvastatin, Spironolactone, Succinylsulphathiazole, Sulphadiazine, Sulphadimethoxine, Sulphadimidine, Sulphafurazole, Sulphaphenazole, Thiabendazole, Timidazole, Tolbutamide, Triamterene, Sulphamethoxazole and the like and their pharmaceutically acceptable salts, ester and hydrates.

74. The pharmaceutical composition of claim 73, wherein the active ingredient is Clarithromycin.

75. The pharmaceutical composition of claim 55, wherein the active pharmaceutical ingredient is a low soluble low dose API or a derivative thereof.

76. The pharmaceutical composition of claim 75, wherein the low soluble low dose API or a derivative is selected from the group consisting of Alprazolam, Amiloride hydrochloride, Astemizole, Atorvastatin, Benzhexol hydrochloride, Betamethasone, Bromhexine hydrochloride, Bromocryptine mesylate, Buprenorphine hydrochloride, carbimazole, Chlordiazepoxide, Citalopram, Cortisone acetate, cyproheptadine hydrochloride, diazepam, Desloratadine, Dexamethasone hydrochloride, Dextromethorphan hydrochloride, dicyclomine hydrochloride, Dienoestrol, Digitoxin, Digoxin, Domperidone, Dydrogesterone, enalapril maleate, Esomeprazole, Ethinyloestradiol, Ethyloestrenol, Fludrocortisone acetate, Frusemide, Glibenclamide, Glimepiride, Haloperidol, Indomethacin, Isoxsuprine hydrochloride, Lanatoside C, Lercanidipine hydrochloride, Levonorgesrtel, Lomustine, Loratadine, Isoxuprine hydrochloride, methotrexate, Mecizine hydrochloride, Melphalan, Methotrexate, Methylergometrine maleate, Methylprednisolone, Mianserin hydrochloride, Mosapride, Nicoumalone, Nifedipine, Nitrazepam, Norethisterone, nortriptyline hydrochloride, Omeprazole, Ormeloxifene hydrochloride, Pentazocine hydrochloride, Phenindamine tartrate, piroxicam, prazosin hydrochloride, Prednisolone, Prednisone, Prochlorperazine maleate, Repaglinide, Riboflavinee, Rosuvastatin, Stilbostrol, Tamoxifen citrate, Thyroxine sodium, triamcinilone, Trimethoprim, Valdecoxib, Zolpidem tartrate and the like and their pharmaceutically acceptable salts, ester and hydrates.

77. The pharmaceutical composition of claim 76, wherein the active ingredient is Alprazolam.

78. The pharmaceutical composition of claim 76, wherein the active ingredient is Lercanidipine hydrochloride.

79. The pharmaceutical composition of claim 55, wherein the primary release modifying agent is a low molecular weight polyethylene oxide.

80. The pharmaceutical composition of claim 55, wherein the low molecular weight polyethylene oxide has a molecular weight of at least about 100,000.

81. The pharmaceutical composition of claim 55, wherein the low molecular weight polyethylene oxide has a molecular weight ranging from 100,000 to 900,000.

82. The pharmaceutical composition of claim 55, wherein the auxiliary release modifying agent is a starch derivative selected from pregelatinized starch, partially pregelatinized starch, retrograded starch, or a combination thereof.

83. The pharmaceutical composition of claim 55, wherein the auxiliary release modifying agent is a retrograded starch.

84. The pharmaceutical composition of claim 55, wherein said composition further comprises at least one pharmaceutically acceptable additive selected from diluents, fillers, binders, glidants, or lubricants.

85. The pharmaceutical composition of claim 55, wherein said composition further comprises an optional coating which is designed for the modification of drug release.

86. The pharmaceutical composition of claim 85, wherein the coating composition is selected from the group consisting of cellulose ethers such as ethyl cellulose, hydroxypropyl methyl cellulose, hydroxypropyl cellulose, others such as polyvinyl alcohol, polyvinyl pyrrolidone, methacrylic acid derivatives, resins, clays, long chain hydrocarbons, long chain carboxylic acids, long chain carboxylic acid esters, long chain alcohols and the like or mixtures thereof.

87. The pharmaceutical composition of claim 55, wherein said composition further comprises an optional coating which is not designed for the modification of drug release.

88. The pharmaceutical composition of claim 87, wherein the coating composition is selected from the group consisting of cellulose ethers such as ethyl cellulose, hydroxypropyl methyl cellulose, hydroxypropyl cellulose, and others such as polyvinyl alcohol, polyvinyl pyrrolidone, methacrylic acid derivatives, resins, clays, long chain hydrocarbons, long chain carboxylic acids, long chain carboxylic acid esters, long chain alcohols and the like and mixtures thereof.

89. The pharmaceutical composition of claim 55, wherein said composition comprises from about 0.1 percent weight to about 90 percent weight of the active pharmaceutical ingredient.

90. The pharmaceutical composition of claim 89, wherein said composition comprises from about 0.1 percent weight to about 80 percent weight of the active pharmaceutical ingredient.

91. The pharmaceutical composition of claim 55, wherein the composition comprises from about 1 percent weight to about 90 percent weight of the release modifying complex.

92. The pharmaceutical composition of claim 91, wherein the composition comprises from about 5 percent weight to about 85 percent weight of the release modifying complex.

93. The pharmaceutical composition of claim 92, wherein the composition comprises from about 10 percent weight to about 80 percent weight of the release modifying complex.

94. The pharmaceutical composition of claim 55, wherein the primary release modifying agent comprises from about 1 percent weight to about 90 percent weight of the release modifying complex.

95. The pharmaceutical composition of claim 94, wherein the primary release modifying agent comprises from about 5 percent weight to about 80 percent weight of the release modifying complex.

96. The pharmaceutical composition of claim 95, wherein the primary release modifying agent comprises from about 5 percent weight to about 70 percent weight of the release modifying complex.

97. The pharmaceutical composition of claim 55, wherein the auxiliary release modifying agent comprises from about 1 percent weight to about 95 percent weight of the release modifying complex.

98. The pharmaceutical composition of claim 97, wherein the auxiliary release modifying agent comprises from about 5 percent weight to about 95 percent weight of the release modifying complex.

99. The pharmaceutical composition of claim 98, wherein the auxiliary release modifying agent comprises from about 10 percent weight to about 95 percent weight of the release modifying complex.

100. A controlled release pharmaceutical composition comprising a pharmaceutically active ingredient and a release modifying complex, wherein said release modifying complex comprises a synergistic combination of (a) a secondary release modifying agent selected from a high molecular weight polyethylene oxide and (b) an auxiliary release modifying agent selected from a starch derivative, and said primary release modifying agent and auxiliary release modifying agent are present in synergistic effective amounts to extend the release of the active pharmaceutical ingredient.

101. The pharmaceutical composition of claim 100, wherein the active pharmaceutical ingredient is a macrolide or azide antibiotic or a derivative thereof.

102. The pharmaceutical composition of claim 101, wherein the macrolide is an erythromycin derivative or its pharmaceutically acceptable hydrates, salts or esters.

103. The pharmaceutical composition of claim 100, wherein the azide is azithromycin, or its pharmaceutically acceptable hydrates, salts or esters.

104. The pharmaceutical composition of claim 102, wherein the erythromycin derivative is selected from the group consisting of clarithromycin, josamycin, midecamycin, kitamycin, roxithromycin, rokitamycin, oleandomycin, miocamycin, flurithromycin, and rosaramicin, and their pharmaceutically acceptable hydrates, salts and esters.

105. The pharmaceutical composition of claim 100, wherein the active pharmaceutical ingredient is a penicillin antibiotic or a derivative thereof.

106. The pharmaceutical composition of claim 105, wherein the penicillin derivative is selected from the group consisting of Amoxicillin, Ampicillin, Ampicillin Sodium, Apalcillin, Aspoxicillin, Azlocillin, Aztreonam, Bacampicillin, Cabenicillin, Carfecillin, Carindacillin, Ciclacillin, Cloxacillin, Dicloxacillin, and their pharmaceutically acceptable hydrates, salts and esters.

107. The pharmaceutical composition of claim 100, wherein the active pharmaceutical ingredient is a cephalosporin antibiotic or a derivative thereof.

108. The pharmaceutical composition of claim 107, wherein the cephalosporin derivative is selected from the group consisting of Cefacetrile, Cefadroxil, Cefaloridine, Cefalothin Sodium, Cefapirin, Cefazaflur, Cefazedone, Cefazolin, Cefradine, Ceftezole, Cefsulodin Sodium, Cefamandole, Cefonicid, Cefoperazone, Cefuroxime, Cefuzonam, Cefbuperazone, Cefoxitin, Cefminox, Cefinetazole, Cefotetan, Loracarbef, Cefinenoxime, Cefodizime Sodium, Cefotaxime, Cefpimizole, Cefpiramide, Ceftazidime, Ceftiolene, Ceftizoxime, Ceftriaxone, Cefepime, Cefetecol, Cefpirome, Cefquinome, Cefalosporin C, Cefozopran Hydrochloride, Cefaclor, Cefadroxil, Cefalexin, Cefaloglycine, Cefatrizine, Cefdinir, Cefetamet Pivoxil, Cefixime, Ceforamide, Cefotiam, Cefpodoxime, Cefpodoxime Proxetil, Cefprozil, Cefradine, Cefroxadine, Cefteram Pivoxil, Ceftibuten, Cefuroxime Axetil, and their pharmaceutically acceptable hydrates, salts and esters.

109. The pharmaceutical composition of claim 100, wherein the active pharmaceutical ingredient is a quinolone antibiotic or a derivative thereof.

110. The pharmaceutical composition of claim 109, wherein the quinolone derivative is selected from the group consisting of Nalidixic Acid, Cinoxacin, Oxolinic Acid, Flumequine, Pipemidic Acid, Rosoxacin, Norfloxacin, Lomefloxacin, Ofloxacin, Enrofloxacin, Ciprofloxacin, Enoxacin, Amifloxacin, Fleroxacin, Gatifloxacin, Gemifloxacin, Pefloxacin, Rufloxacin, Sparfloxacin, Temafloxacin, Tosufloxacin, Grepafloxacin, Levofloxacin, Moxifloxacin, Trovafloxacin, and their pharmaceutically acceptable hydrates, salts and esters.

111. The pharmaceutical composition of claim 100, wherein the active pharmaceutical ingredient is a high soluble high dose API or a derivative thereof.

112. The pharmaceutical composition of claim 111, wherein the high soluble high dose API or a derivative is selected from the group consisting of Acebutolol hydrochloride, Amantadine hydrochloride, Aminocaproic acid, Aminophylline, Amodiaquine hydrochloride, Ascorbic acid, Bupropion hydrochloride, Carbenoxolone sodium, Cefuroxime sodium, Chloroquine phosphate, Chloroquine sulphate, Chlorpromazine hydrochloride, Ciprofloxacin hydrochloride, Cloxacillin sodium, Cycloserine, Diltiazem hydrochloride, Diethyl carbamazine citrate, Doxycycline hydrochloride, Ethosuximide, Ferrous gluconate, Isoniazid, Levamisole hydrochloride, Levetiracetam, Lincomycin hydrochloride, Mebeverine hydrochloride, Mepyramine maleate, Metformin hydrochloride, Metoprolol tartrate, Metoprolol succinate, Niacin, Nicotinamide, Nicotinic acid, Oxprenolol hydrochloride, oxytetracycline hydrochloride, Penicillamine, Pentobarbitone sodium, Phenoxy Methyl Penicillin K, Phenyloin sodium, piperazine adipate, Potassium chloride, Procainamide hydrochloride, Propranolol hydrochloride, Pseudoephedrine hydrochloride, Quinalbarbitone sodium, Quinine bisulphate, Ranitidine hydrochloride, Sodium amino salicylate, Sodium fusidate, sodium valproate, Streptomycin sulphate, Tetracycline hydrochloride, Topiramate, Troxidone, Verapamil hydrochloride and the like and their pharmaceutically acceptable salts, ester and hydrates.

113. The pharmaceutical composition of claim 112, wherein the active ingredient is Nicotinic acid.

114. The pharmaceutical composition of claim 100, wherein the active pharmaceutical ingredient is a high soluble low dose API or a derivative thereof.

115. The pharmaceutical composition of claim 114, wherein the high soluble low dose API or a derivative is selected from the group consisting of Amitriptylline hydrochloride, captopril, Clonidine hydrochloride, Colchicin, Cyclophosphamide, Diphenhydramine hydrochloride, Dothiepen hydrochloride, Doxepin hydrochloride Ephedrine hydrochloride, Ergometrine maleate, Ergometrine tartrate, Fenfluramine hydrochloride, Folic acid, Glyceryl trinitrate, Hyoscine hydrobromide, Hyoscine buytlbromide, Imipramine hydrochloride, Isoprenaline sulphate, Isosorbide dinitrate, Isosorbide-5-mononitrate, Levocetrizine hydrochloride, Methadone hydrochloride, Methdilazine hydrochloride, Metoclopramide hydrochloride, Neostigmine bromide, Oxybutynin hydrochloride, Perindopril erbumine, Pethidine hydrochloride, Phenformin hydrochloride, Pheniramine maleate, Phenobarbitone sodium, Primaquine phosphate, Promethazine hydrochloride, Propantheline bromide, Propranolol hydrochloride, Pyridoxine hydrochloride, Salbutamol sulphate, Terbutaline sulphate, Thiamine hydrochloride, Timolol maleate, Tizanidine hydrochloride, Trifluoperazine hydrochloride, Triflupromazine hydrochloride, Triprolidine hydrochloride, Warfarin sodium and the like and their pharmaceutically acceptable salts, ester and hydrates.

116. The pharmaceutical composition of claim 115, wherein the active ingredient is Oxybutynin hydrochloride.

117. The pharmaceutical composition of claim 100, wherein the active pharmaceutical ingredient is a low soluble high dose API or a derivative thereof.

118. The pharmaceutical composition of claim 117, wherein the low soluble high dose API or a derivative is selected from the group consisting of Acetazolamide, Allopurinol, Atenolol, Carbamazepine, Cefadroxil, Cephalexin, Chloramphenicol, Cefuroxime Axetil, Chlorthalidone, Cilastozol, Cimetidine, Clarithromycin, Clofazemine, Dapsone, Diclofenac sodium, Diiodohydroxy quinolone, Diloxamide furoate, Disulfiram, Erythromycin, Erythromycin estolate, Erythromycin stearate, Ethacrynic cid, Ethionamide, Ethopropazine hydrochloride, Ferrous fumarate, Fluconazole, Flurbiprofen, Furazolidone, Griseofulvin, Hydrochlorthiazide, Ibuprofen, Itraconazole, Ketoconazole, Ketoprofen, Labetalol hydrochloride, Levodopa, Linezolid, Lithium carbonate, Magaldrate, Mebendazole, Mefenamic acid, Megestrol acetate, Mercaptopurine, Mesalamimne, Nalidixic acid, Nateglinide, Niclosamide, Nitrofurantoin, Norfloxacin, Oxcarbazepine, Oxyphenbutazone, Paracetamol, Phenindione, Phenobarbitone, Phenylbutazone, Phenylsulphathiazole, piperazine phosphate, Proguanil hydrochloride, Promethazine theoclate, Propylthiouracil, Quinidine sulphate, Quinine sulphate, Quinidochlor, Rifampicin, Simvastatin, Spironolactone, Succinylsulphathiazole, Sulphadiazine, Sulphadimethoxine, Sulphadimidine, Sulphafurazole, Sulphaphenazole, Thiabendazole, Timidazole, Tolbutamide, Triamterene, Sulphamethoxazole and the like and their pharmaceutically acceptable salts, ester and hydrates.

119. The pharmaceutical composition of claim 118, wherein the active ingredient is Clarithromycin.

120. The pharmaceutical composition of claim 100, wherein the active pharmaceutical ingredient is a low soluble low dose API or a derivative thereof.

121. The pharmaceutical composition of claim 120, wherein the low soluble low dose API or a derivative is selected from the group consisting of Alprazolam, Amiloride hydrochloride, Astemizole, Atorvastatin, Benzhexol hydrochloride, Betamethasone, Bromhexine hydrochloride, Bromocryptine mesylate, Buprenorphine hydrochloride, carbimazole, Chlordiazepoxide, Citalopram, Cortisone acetate, cyproheptadine hydrochloride, diazepam, Desloratadine, Dexamethasone hydrochloride, Dextromethorphan hydrochloride, dicyclomine hydrochloride, Dienoestrol, Digitoxin, Digoxin, Domperidone, Dydrogesterone, enalapril maleate, Esomeprazole, Ethinyloestradiol, Ethyloestrenol, Fludrocortisone acetate, Frusemide, glibenclamide, Glimepiride, haloperidol, Indomethacin, Isoxsuprine hydrochloride, Lanatoside C, Lercanidipine hydrochloride, Levonorgesrtel, Lomustine, Loratadine, Isoxuprine hydrochloride, methotrexate, Mecizine hydrochloride, Melphalan, Methotrexate, Methylergometrine maleate, Methylprednisolone, Mianserin hydrochloride, Mosapride, Nicoumalone, Nifedipine, Nitrazepam, Norethisterone, nortriptyline hydrochloride, Omeprazole, Ormeloxifene hydrochloride, Pentazocine hydrochloride, Phenindamine tartrate, piroxicam, prazosin hydrochloride, Prednisolone, Prednisone, Prochlorperazine maleate, Repaglinide, Riboflavinee, Rosuvastatin, Stilbostrol, Tamoxifen citrate, Thyroxine sodium, triamcinilone, Trimethoprim, Valdecoxib, Zolpidem tartrate and the like and their pharmaceutically acceptable salts, ester and hydrates.

122. The pharmaceutical composition of claim 121, wherein the active ingredient is Alprazolam.

123. The pharmaceutical composition of claim 121, wherein the active ingredient is Lercanidipine hydrochloride.

124. The pharmaceutical composition of claim 100, wherein the secondary release modifying agent is a high molecular weight polyethylene oxide.

125. The pharmaceutical composition of claim 100, wherein the high molecular weight polyethylene oxide has a molecular weight of at least about 1,000,000.

126. The pharmaceutical composition of claim 100, wherein the high molecular weight polyethylene oxide has a molecular weight ranging from 1,000,000 to 9,000,000.

127. The pharmaceutical composition of claim 100, wherein the auxiliary release modifying agent is a starch derivative selected from pregelatinized starch, partially pregelatinized starch, retrograded starch, or a combination thereof.

128. The pharmaceutical composition of claim 100, wherein the auxiliary release modifying agent is a retrograded starch.

129. The pharmaceutical composition of claim 100, wherein said composition further comprises at least one pharmaceutically acceptable additive selected from diluents, fillers, binders, glidants, and lubricants.

130. The pharmaceutical composition of claim 100, wherein said composition further comprises an optional coating which is designed for the modification of drug release.

131. The pharmaceutical composition of claim 130, wherein the coating composition is selected from the group consisting of cellulose ethers such as ethyl cellulose, hydroxypropyl methyl cellulose, hydroxypropyl cellulose, others such as polyvinyl alcohol, polyvinyl pyrrolidone, methacrylic acid derivatives, resins, clays, long chain hydrocarbons, long chain carboxylic acids, long chain carboxylic acid esters, long chain alcohols and the like and mixtures thereof.

132. The pharmaceutical composition of claim 100, wherein said composition further comprises an optional coating which is not designed for the modification of drug release.

133. The pharmaceutical composition of claim 132, wherein the coating composition is selected from the group consisting of cellulose ethers such as ethyl cellulose, hydroxypropyl methyl cellulose, hydroxypropyl cellulose, and others such as polyvinyl alcohol, polyvinyl pyrrolidone, methacrylic acid derivatives, resins, clays, long chain hydrocarbons, long chain carboxylic acids, long chain carboxylic acid esters, long chain alcohols and the like and mixtures thereof.

134. The pharmaceutical composition of claim 100, wherein said composition comprises from about 0.1 percent weight to about 90 percent weight of the active pharmaceutical ingredient.

135. The pharmaceutical composition of claim 134, wherein said composition comprises from about 0.1 percent weight to about 80 percent weight of the active pharmaceutical ingredient.

136. The pharmaceutical composition of claim 100, wherein the composition comprises from about 1 percent weight to about 90 percent weight of the release modifying complex.

137. The pharmaceutical composition of claim 136, wherein the composition comprises from about 5 percent weight to about 85 percent weight of the release modifying complex.

138. The pharmaceutical composition of claim 137, wherein the composition comprises from about 10 percent weight to about 80 percent weight of the release modifying complex.

139. The pharmaceutical composition of claim 100, wherein the secondary release modifying agent comprises from about 1 percent weight to about 95 percent weight of the release modifying complex.

140. The pharmaceutical composition of claim 139, wherein the secondary release modifying agent comprises from about 5 percent weight to about 90 percent weight of the release modifying complex.

141. The pharmaceutical composition of claim 100, wherein the auxiliary release modifying agent comprises from about 1 percent weight to about 95 percent weight of the release modifying complex.

142. The pharmaceutical composition of claim 141, wherein the auxiliary release modifying agent comprises from about 5 percent weight to about 95 percent weight of the release modifying complex.

143. The pharmaceutical composition of claim 142, wherein the auxiliary release modifying agent comprises from about 10 percent weight to about 95 percent weight of the release modifying complex.

144. A process for the preparation of controlled release pharmaceutical composition of the present invention.

145. A pharmaceutical composition which gives an improved method of extended release of the API from the dosage form, comprising of the API, the release retarding complex and the other required pharmaceutically acceptable additives; the improvements comprising the synergistic combination of release retardant complex in synergistic effective amounts to extend the release of the API.
